SEQUENCE LISTING (123 sequences)

SEQ ID NO: 1 - protein, 15 aa, synthetic construct
  REGION 1..15: note = Description of Artificial Sequence: Synthetic peptide
CTTSGFNIKDTYIHW

SEQ ID NO: 2 - protein, 13 aa, synthetic construct
  REGION 1..13: note = Description of Artificial Sequence: Synthetic peptide
GRNDPANGNTKYD

SEQ ID NO: 3 - protein, 14 aa, synthetic construct
  REGION 1..14: note = Description of Artificial Sequence: Synthetic peptide
CASYEGYYERFGYW

SEQ ID NO: 4 - protein, 18 aa, synthetic construct
  REGION 1..18: note = Description of Artificial Sequence: Synthetic peptide
CKSSQSLLDRDGKTYLNW

SEQ ID NO: 5 - protein, 7 aa, synthetic construct
  REGION 1..7: note = Description of Artificial Sequence: Synthetic peptide
LVSKLDS

SEQ ID NO: 6 - protein, 11 aa, synthetic construct
  REGION 1..11: note = Description of Artificial Sequence: Synthetic peptide
CWQDTHFPYTF

SEQ ID NO: 7 - protein, 119 aa, synthetic construct
  REGION 1..119: note = Description of Artificial Sequence: Synthetic polypeptide
EVQLQQSGAELVNPGASVKLSCTTSGFNIKDTYIHWVKQRPEQGLEWIGRNDPANGNTKYDPKFQGKATITADTSSSTAYLQLSSLTSEDTAVYYCASYEGYYERFGYWGQGTLVTVSA

SEQ ID NO: 8 - protein, 112 aa, synthetic construct
  REGION 1..112: note = Description of Artificial Sequence: Synthetic polypeptide
DVVMTQAPLTLSVTIGQPASISCKSSQSLLDRDGKTYLNWLLQRPGQSPKRLIYLVSKLDSGVPDRFTGSGSGTDFTLKISRVEAEDLGVYYCWQDTHFPYTFGGGTKLEIK

SEQ ID NO: 9 - protein, 119 aa, synthetic construct
  REGION 1..119: note = Description of Artificial Sequence: Synthetic polypeptide
QVQLVQSGAEVKKPGSSVKVSCKTSGFNIKDTYIHWVRQAPGQGLEWIGRNDPANGNTKYAQKFQGRATITADESTSTAYMELSSLRSEDTAVYYCASYEGYYERFGYWGQGTLVTVSS

SEQ ID NO: 10 - protein, 112 aa, synthetic construct
  REGION 1..112: note = Description of Artificial Sequence: Synthetic polypeptide
DIQMTQSPSSLSASVGDRVTITCRSSQSLLDRDGKSYLNWLQQKPGKAPKRLIYLVSKLDSGVPSRFSGSGSGTDFTLTISSLQPEDFATYYCWQDTHFPYTFGQGTKLEIK

SEQ ID NO: 11 - protein, 119 aa, synthetic construct
  REGION 1..119: note = Description of Artificial Sequence: Synthetic polypeptide
QVQLVQSGAEVKKPGASVKVSCKTSGFNIKDTYIHWVRQAPGQGLEWIGRNDPANGNTKYAQKFQGRVTMTADTSTSTVYMELSSLRSEDTAVYYCASYEGYYERFGYWGQGTLVTVSS

SEQ ID NO: 12 - protein, 112 aa, synthetic construct; identical to SEQ ID NO: 10
  REGION 1..112: note = Description of Artificial Sequence: Synthetic polypeptide

SEQ ID NO: 13 - protein, 119 aa, synthetic construct
  REGION 1..119: note = Description of Artificial Sequence: Synthetic polypeptide
EVQLVESGGGLVQPGRSLRLSCTTSGFNIKDTYIHWFRQAPGKGLEWVGRNDPANGNTKYTASFKGRATISADGSKSIAYLQMNSLKTEDTAVYYCASYEGYYERFGYWGQGTLVTVSS

SEQ ID NO: 14 - protein, 112 aa, synthetic construct; identical to SEQ ID NO: 10
  REGION 1..112: note = Description of Artificial Sequence: Synthetic polypeptide

SEQ ID NO: 15 - protein, 119 aa, synthetic construct
  REGION 1..119: note = Description of Artificial Sequence: Synthetic polypeptide
EVQLVESGGGLVQPGGSLKLSCATSGFNIKDTYIHWVRQASGKGLEWIGRNDPANGNTKYAASFKGRATISADDSKNTAYLQLNSLKTEDTAVYYCASYEGYYERFGYWGQGTLVTVSS

SEQ ID NO: 16 - protein, 112 aa, synthetic construct; identical to SEQ ID NO: 10
  REGION 1..112: note = Description of Artificial Sequence: Synthetic polypeptide

SEQ ID NO: 17 - protein, 119 aa, synthetic construct
  REGION 1..119: note = Description of Artificial Sequence: Synthetic polypeptide
QVQLQESGPGLVKPSGTLSLTCAVSGFNIKDTYIHWVRQPPGKGLEWIGRNDPANGNTKYNPSFKSRATISADKSKNQASLKLSSVTAADTAVYYCASYEGYYERFGYWGQGTLVTVSS

SEQ ID NO: 18 - protein, 112 aa, synthetic construct; identical to SEQ ID NO: 10
  REGION 1..112: note = Description of Artificial Sequence: Synthetic polypeptide

SEQ ID NO: 19 - protein, 119 aa, synthetic construct
  REGION 1..119: note = Description of Artificial Sequence: Synthetic polypeptide
EVQLVQSGAEVKKPGESLKISCKTSGFNIKDTYIHWVRQMPGKGLEWIGRNDPANGNTKYSPSFQGQVTISADKSISTAYLQLSSLKASDTAMYYCASYEGYYERFGYWGQGTLVTVSS

SEQ ID NO: 20 - protein, 112 aa, synthetic construct; identical to SEQ ID NO: 10
  REGION 1..112: note = Description of Artificial Sequence: Synthetic polypeptide

SEQ ID NO: 21 - protein, 119 aa, synthetic construct; identical to SEQ ID NO: 9
  REGION 1..119: note = Description of Artificial Sequence: Synthetic polypeptide

SEQ ID NO: 22 - protein, 112 aa, synthetic construct
  REGION 1..112: note = Description of Artificial Sequence: Synthetic polypeptide
DVVMTQSPLSLPVTLGQPASISCRSSQSLLDRDGKTYLNWLQQRPGQSPRRLIYLVSKRDSGVPDRFSGSGSGTDFTLKISRVEAEDVGVYYCWQDTHFPYTFGQGTKLEIK

SEQ ID NO: 23 - protein, 119 aa, synthetic construct; identical to SEQ ID NO: 11
  REGION 1..119: note = Description of Artificial Sequence: Synthetic polypeptide

SEQ ID NO: 24 - protein, 112 aa, synthetic construct; identical to SEQ ID NO: 22
  REGION 1..112: note = Description of Artificial Sequence: Synthetic polypeptide

SEQ ID NO: 25 - protein, 119 aa, synthetic construct; identical to SEQ ID NO: 13
  REGION 1..119: note = Description of Artificial Sequence: Synthetic polypeptide

SEQ ID NO: 26 - protein, 112 aa, synthetic construct; identical to SEQ ID NO: 22
  REGION 1..112: note = Description of Artificial Sequence: Synthetic polypeptide

SEQ ID NO: 27 - protein, 119 aa, synthetic construct; identical to SEQ ID NO: 15
  REGION 1..119: note = Description of Artificial Sequence: Synthetic polypeptide

SEQ ID NO: 28 - protein, 112 aa, synthetic construct; identical to SEQ ID NO: 22
  REGION 1..112: note = Description of Artificial Sequence: Synthetic polypeptide

SEQ ID NO: 29 - protein, 119 aa, synthetic construct; identical to SEQ ID NO: 17
  REGION 1..119: note = Description of Artificial Sequence: Synthetic polypeptide

SEQ ID NO: 30 - protein, 112 aa, synthetic construct; identical to SEQ ID NO: 22
  REGION 1..112: note = Description of Artificial Sequence: Synthetic polypeptide

SEQ ID NO: 31 - protein, 119 aa, synthetic construct; identical to SEQ ID NO: 19
  REGION 1..119: note = Description of Artificial Sequence: Synthetic polypeptide

SEQ ID NO: 32 - protein, 112 aa, synthetic construct; identical to SEQ ID NO: 22
  REGION 1..112: note = Description of Artificial Sequence: Synthetic polypeptide

SEQ ID NO: 33 - protein, 119 aa, synthetic construct; identical to SEQ ID NO: 9
  REGION 1..119: note = Description of Artificial Sequence: Synthetic polypeptide

SEQ ID NO: 34 - protein, 112 aa, synthetic construct
  REGION 1..112: note = Description of Artificial Sequence: Synthetic polypeptide
EIVLTQSPGTLSLSPGERATLSCRSSQSLLDRDGKTYLNWLQQKPGQAPRRLIYLVSKLDSGIPDRFSGSGSGTDFTLTISRLEPEDFAVYYCWQDTHFPYTFGQGTKLEIK

SEQ ID NO: 35 - protein, 119 aa, synthetic construct; identical to SEQ ID NO: 11
  REGION 1..119: note = Description of Artificial Sequence: Synthetic polypeptide

SEQ ID NO: 36 - protein, 112 aa, synthetic construct; identical to SEQ ID NO: 34
  REGION 1..112: note = Description of Artificial Sequence: Synthetic polypeptide

SEQ ID NO: 37 - protein, 119 aa, synthetic construct; identical to SEQ ID NO: 13
  REGION 1..119: note = Description of Artificial Sequence: Synthetic polypeptide

SEQ ID NO: 38 - protein, 112 aa, synthetic construct; identical to SEQ ID NO: 34
  REGION 1..112: note = Description of Artificial Sequence: Synthetic polypeptide

SEQ ID NO: 39 - protein, 119 aa, synthetic construct; identical to SEQ ID NO: 15
  REGION 1..119: note = Description of Artificial Sequence: Synthetic polypeptide

SEQ ID NO: 40 - protein, 112 aa, synthetic construct; identical to SEQ ID NO: 34
  REGION 1..112: note = Description of Artificial Sequence: Synthetic polypeptide

SEQ ID NO: 41 - protein, 119 aa, synthetic construct; identical to SEQ ID NO: 17
  REGION 1..119: note = Description of Artificial Sequence: Synthetic polypeptide

SEQ ID NO: 42 - protein, 112 aa, synthetic construct; identical to SEQ ID NO: 34
  REGION 1..112: note = Description of Artificial Sequence: Synthetic polypeptide

SEQ ID NO: 43 - protein, 119 aa, synthetic construct; identical to SEQ ID NO: 19
  REGION 1..119: note = Description of Artificial Sequence: Synthetic polypeptide

SEQ ID NO: 44 - protein, 112 aa, synthetic construct; identical to SEQ ID NO: 34
  REGION 1..112: note = Description of Artificial Sequence: Synthetic polypeptide

SEQ ID NO: 45 - protein, 119 aa, synthetic construct; identical to SEQ ID NO: 9
  REGION 1..119: note = Description of Artificial Sequence: Synthetic polypeptide

SEQ ID NO: 46 - protein, 112 aa, synthetic construct
  REGION 1..112: note = Description of Artificial Sequence: Synthetic polypeptide
DIVMTQSPDSLAVSLGERATINCKSSQSLLDRDGKTYLNWLQQKPGQSPKRLIYLVSKLDSGVPDRFSGSGSGTDFTLTISSLQAEDLAVYYCWQDTHFPYTFGQGTKLEIK

SEQ ID NO: 47 - protein, 119 aa, synthetic construct; identical to SEQ ID NO: 11
  REGION 1..119: note = Description of Artificial Sequence: Synthetic polypeptide

SEQ ID NO: 48 - protein, 112 aa, synthetic construct; identical to SEQ ID NO: 46
  REGION 1..112: note = Description of Artificial Sequence: Synthetic polypeptide

SEQ ID NO: 49 - protein, 119 aa, synthetic construct; identical to SEQ ID NO: 13
  REGION 1..119: note = Description of Artificial Sequence: Synthetic polypeptide

SEQ ID NO: 50 - protein, 112 aa, synthetic construct; identical to SEQ ID NO: 46
  REGION 1..112: note = Description of Artificial Sequence: Synthetic polypeptide

SEQ ID NO: 51 - protein, 119 aa, synthetic construct; identical to SEQ ID NO: 15
  REGION 1..119: note = Description of Artificial Sequence: Synthetic polypeptide

SEQ ID NO: 52 - protein, 112 aa, synthetic construct; identical to SEQ ID NO: 46
  REGION 1..112: note = Description of Artificial Sequence: Synthetic polypeptide

SEQ ID NO: 53 - protein, 119 aa, synthetic construct; identical to SEQ ID NO: 17
  REGION 1..119: note = Description of Artificial Sequence: Synthetic polypeptide

SEQ ID NO: 54 - protein, 112 aa, synthetic construct; identical to SEQ ID NO: 46
  REGION 1..112: note = Description of Artificial Sequence: Synthetic polypeptide

SEQ ID NO: 55 - protein, 119 aa, synthetic construct; identical to SEQ ID NO: 19
  REGION 1..119: note = Description of Artificial Sequence: Synthetic polypeptide

SEQ ID NO: 56 - protein, 112 aa, synthetic construct; identical to SEQ ID NO: 46
  REGION 1..112: note = Description of Artificial Sequence: Synthetic polypeptide

SEQ ID NO: 57 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 107 aa, Homo sapiens [sequence omitted]

SEQ ID NO: 59 - protein, 449 aa, synthetic construct
  REGION 1..449: note = Description of Artificial Sequence: Synthetic polypeptide
QVQLVQSGAEVKKPGSSVKVSCKTSGFNIKDTYIHWVRQAPGQGLEWIGRNDPANGNTKYAQKFQGRATITADESTSTAYMELSSLRSEDTAVYYCASYEGYYERFGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 219 aa, synthetic construct
  REGION 1..219: note = Description of Artificial Sequence: Synthetic polypeptide
DIQMTQSPSSLSASVGDRVTITCRSSQSLLDRDGKSYLNWLQQKPGKAPKRLIYLVSKLDSGVPSRFSGSGSGTDFTLTISSLQPEDFATYYCWQDTHFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 61 - protein, 15 aa, synthetic construct
  REGION 1..15: note = Description of Artificial Sequence: Synthetic peptide
CTASGVNIKDTYMYW

SEQ ID NO: 62 - protein, 13 aa, synthetic construct
  REGION 1..13: note = Description of Artificial Sequence: Synthetic peptide
GRIDPANGYTRYD

SEQ ID NO: 63 - protein, 14 aa, synthetic construct
  REGION 1..14: note = Description of Artificial Sequence: Synthetic peptide
CAVYDGYYESFDVW

SEQ ID NO: 64 - protein, 18 aa, synthetic construct
  REGION 1..18: note = Description of Artificial Sequence: Synthetic peptide
CKSSQSLLDSDGKTYLNW

SEQ ID NO: 65 - protein, 7 aa, synthetic construct
  REGION 1..7: note = Description of Artificial Sequence: Synthetic peptide
LVSKLDS

SEQ ID NO: 66 - protein, 11 aa, synthetic construct
  REGION 1..11: note = Description of Artificial Sequence: Synthetic peptide
CWQGTLFPRTF

SEQ ID NO: 67 - protein, 119 aa, synthetic construct
  REGION 1..119: note = Description of Artificial Sequence: Synthetic polypeptide
EVQLQQSGAELVKPGASVKLSCTASGVNIKDTYMYWVKQRPEQGLEWIGRIDPANGYTRYDPKFQGKATITADTSSNTAHLQLSSLTSEDAAVYYCAVYDGYYESFDVWGAGTTVTVSS

SEQ ID NO: 68 - protein, 112 aa, synthetic construct
  REGION 1..112: note = Description of Artificial Sequence: Synthetic polypeptide
DVVMTQAPLTLSVTIGQPASISCKSSQSLLDSDGKTYLNWLLQRPGQSPKRLIYLVSKLDSGVPDRFTGSGSGTDFTLKINRVEAEDLGVYYCWQGTLFPRTFGGGTKLEIK

SEQ ID NO: 69 - protein, 119 aa, synthetic construct
  REGION 1..119: note = Description of Artificial Sequence: Synthetic polypeptide
QVQLVQSGAEVKKPGSSVKVSCKASGVNIKDTYMYWVRQAPGQGLEWIGRIDPANGYARYAQKFQGRATITADESTSTAYMELSSLRSEDTAVYYCAVYDGYYESFDVWGQGTLVTVSS

SEQ ID NO: 70 - protein, 112 aa, synthetic construct
  REGION 1..112: note = Description of Artificial Sequence: Synthetic polypeptide
DIQMTQSPSSLSASVGDRVTITCRSSQSLLDSDGKSYLNWLQQKPGKAPKRLIYLVSKLDSGVPSRFSGSGSGTDFTLTISSLQPEDFATYYCWQGTLFPRTFGQGTKLEIK

SEQ ID NO: 71 - protein, 119 aa, synthetic construct
  REGION 1..119: note = Description of Artificial Sequence: Synthetic polypeptide
QVQLVQSGAEVKKPGASVKVSCKASGVNIKDTYMYWVRQAPGQGLEWIGRIDPANGYTRYAQKFQGRATITADTSTSTVYMELSSLRSEDTAVYYCAVYDGYYESFDVWGQGTLVTVSS

SEQ ID NO: 72 - protein, 112 aa, synthetic construct; identical to SEQ ID NO: 70
  REGION 1..112: note = Description of Artificial Sequence: Synthetic polypeptide

SEQ ID NO: 73 - protein, 119 aa, synthetic construct
  REGION 1..119: note = Description of Artificial Sequence: Synthetic polypeptide
EVQLVESGGGLVQPGRSLRLSCTASGVNIKDTYMYWVRQAPGKGLEWVGRIDPANGYTRYTASFKGRATISADGSKSIAYLQMNSLKTEDTAVYYCAVYDGYYESFDVWGQGTLVTVSS

SEQ ID NO: 74 - protein, 112 aa, synthetic construct; identical to SEQ ID NO: 70
  REGION 1..112: note = Description of Artificial Sequence: Synthetic polypeptide

SEQ ID NO: 75 - protein, 119 aa, synthetic construct
  REGION 1..119: note = Description of Artificial Sequence: Synthetic polypeptide
EVQLVESGGGLVQPGGSLKLSCAASGVNIKDTYMYWVRQASGKGLEWIGRIDPANGYTRYAASFKGRATISADDSKNTAYLQLNSLKTEDTAVYYCAVYDGYYESFDVWGQGTLVTVSS

SEQ ID NO: 76 - protein, 112 aa, synthetic construct; identical to SEQ ID NO: 70
  REGION 1..112: note = Description of Artificial Sequence: Synthetic polypeptide

SEQ ID NO: 77 - protein, 119 aa, synthetic construct
  REGION 1..119: note = Description of Artificial Sequence: Synthetic polypeptide
QVQLQESGPGLVKPSGTLSLTCAASGVNIKDTYMYWVRQPPGKGLEWIGRIDPANGYTRYNPSFKSRATISADKSKNQASLKLSSVTAADTAVYYCAVYDGYYESFDVWGQGTLVTVSS

SEQ ID NO: 78 - protein, 112 aa, synthetic construct; identical to SEQ ID NO: 70
  REGION 1..112: note = Description of Artificial Sequence: Synthetic polypeptide

SEQ ID NO: 79 - protein, 119 aa, synthetic construct
  REGION 1..119: note = Description of Artificial Sequence: Synthetic polypeptide
EVQLVQSGAEVKKPGESLKISCKGSGVNIKDTYMYWVRQMPGKGLEWIGRIDPANGYTRYSPSFQGQVTISADKSISTAYLQLSSLKASDTAMYYCAVYDGYYESFDVWGQGTLVTVSS

SEQ ID NO: 80 - protein, 112 aa, synthetic construct; identical to SEQ ID NO: 70
  REGION 1..112: note = Description of Artificial Sequence: Synthetic polypeptide

SEQ ID NO: 81 - protein, 119 aa, synthetic construct; identical to SEQ ID NO: 69
  REGION 1..119: note = Description of Artificial Sequence: Synthetic polypeptide

SEQ ID NO: 82 - protein, 112 aa, synthetic construct
  REGION 1..112: note = Description of Artificial Sequence: Synthetic polypeptide
DVVMTQSPLSLPVTLGQPASISCRSSQSLLDSDGKTYLNWLQQRPGQSPRRLIYLVSKRDSGVPDRFSGSGSGTDFTLKISRVEAEDLGVYYCWQGTLFPRTFGQGTKLEIK

SEQ ID NO: 83 - protein, 119 aa, synthetic construct; identical to SEQ ID NO: 71
  REGION 1..119: note = Description of Artificial Sequence: Synthetic polypeptide

SEQ ID NO: 84 - protein, 112 aa, synthetic construct; identical to SEQ ID NO: 82
  REGION 1..112: note = Description of Artificial Sequence: Synthetic polypeptide

SEQ ID NO: 85 - protein, 119 aa, synthetic construct; identical to SEQ ID NO: 73
  REGION 1..119: note = Description of Artificial Sequence: Synthetic polypeptide

SEQ ID NO: 86 - protein, 112 aa, synthetic construct; identical to SEQ ID NO: 82
  REGION 1..112: note = Description of Artificial Sequence: Synthetic polypeptide

SEQ ID NO: 87 - protein, 119 aa, synthetic construct; identical to SEQ ID NO: 75
  REGION 1..119: note = Description of Artificial Sequence: Synthetic polypeptide

SEQ ID NO: 88 - protein, 112 aa, synthetic construct; identical to SEQ ID NO: 82
  REGION 1..112: note = Description of Artificial Sequence: Synthetic polypeptide

SEQ ID NO: 89 - protein, 119 aa, synthetic construct; identical to SEQ ID NO: 77
  REGION 1..119: note = Description of Artificial Sequence: Synthetic polypeptide

SEQ ID NO: 90 - protein, 112 aa, synthetic construct; identical to SEQ ID NO: 82
  REGION 1..112: note = Description of Artificial Sequence: Synthetic polypeptide

SEQ ID NO: 91 - protein, 119 aa, synthetic construct; identical to SEQ ID NO: 79
  REGION 1..119: note = Description of Artificial Sequence: Synthetic polypeptide

SEQ ID NO: 92 - protein, 112 aa, synthetic construct; identical to SEQ ID NO: 82
  REGION 1..112: note = Description of Artificial Sequence: Synthetic polypeptide

SEQ ID NO: 93 - protein, 119 aa, synthetic construct; identical to SEQ ID NO: 69
  REGION 1..119: note = Description of Artificial Sequence: Synthetic polypeptide

SEQ ID NO: 94 - protein, 112 aa, synthetic construct
  REGION 1..112: note = Description of Artificial Sequence: Synthetic polypeptide
EIVLTQSPGTLSVSPGERATISCRSSQSLLDSDGKTYLNWLQQKPGQAPRRLIYLVSKRDTGVPDRFSGSGSGTDFTLTISRVEPEDFAVYYCWQGTLFPRTFGQGTKLEIK

SEQ ID NO: 95 - protein, 119 aa, synthetic construct; identical to SEQ ID NO: 71
  REGION 1..119: note = Description of Artificial Sequence: Synthetic polypeptide

SEQ ID NO: 96 - protein, 112 aa, synthetic construct; identical to SEQ ID NO: 94
  REGION 1..112: note = Description of Artificial Sequence: Synthetic polypeptide

SEQ ID NO: 97 - protein, 119 aa, synthetic construct; identical to SEQ ID NO: 73
  REGION 1..119: note = Description of Artificial Sequence: Synthetic polypeptide

SEQ ID NO: 98 - protein, 112 aa, synthetic construct; identical to SEQ ID NO: 94
  REGION 1..112: note = Description of Artificial Sequence: Synthetic polypeptide

SEQ ID NO: 99 - protein, 119 aa, synthetic construct; identical to SEQ ID NO: 75
  REGION 1..119: note = Description of Artificial Sequence: Synthetic polypeptide

SEQ ID NO: 100 - protein, 112 aa, synthetic construct; identical to SEQ ID NO: 94
  REGION 1..112: note = Description of Artificial Sequence: Synthetic polypeptide

SEQ ID NO: 101 - protein, 119 aa, synthetic construct; identical to SEQ ID NO: 77
  REGION 1..119: note = Description of Artificial Sequence: Synthetic polypeptide

SEQ ID NO: 102 - protein, 112 aa, synthetic construct; identical to SEQ ID NO: 94
  REGION 1..112: note = Description of Artificial Sequence: Synthetic polypeptide

SEQ ID NO: 103 - protein, 119 aa, synthetic construct; identical to SEQ ID NO: 79
  REGION 1..119: note = Description of Artificial Sequence: Synthetic polypeptide

SEQ ID NO: 104 - protein, 112 aa, synthetic construct; identical to SEQ ID NO: 94
  REGION 1..112: note = Description of Artificial Sequence: Synthetic polypeptide

SEQ ID NO: 105 - protein, 119 aa, synthetic construct; identical to SEQ ID NO: 69
  REGION 1..119: note = Description of Artificial Sequence: Synthetic polypeptide

SEQ ID NO: 106 - protein, 112 aa, synthetic construct
  REGION 1..112: note = Description of Artificial Sequence: Synthetic polypeptide
DIVMTQSPDSLAVSLGERATINCKSSQSLLDSDGKTYLNWLQQKPGQSPKRLIYLVSKLDSGVPDRFSGSGSGTDFTLTISSVQAEDLAVYYCWQGTLFPRTFGQGTKLEIK

SEQ ID NO: 107 - protein, 119 aa, synthetic construct; identical to SEQ ID NO: 71
  REGION 1..119: note = Description of Artificial Sequence: Synthetic polypeptide

SEQ ID NO: 108 - protein, 112 aa, synthetic construct; identical to SEQ ID NO: 106
  REGION 1..112: note = Description of Artificial Sequence: Synthetic polypeptide

SEQ ID NO: 109 - protein, 119 aa, synthetic construct; identical to SEQ ID NO: 73
  REGION 1..119: note = Description of Artificial Sequence: Synthetic polypeptide

SEQ ID NO: 110 - protein, 112 aa, synthetic construct; identical to SEQ ID NO: 106
  REGION 1..112: note = Description of Artificial Sequence: Synthetic polypeptide

SEQ ID NO: 111 - protein, 119 aa, synthetic construct; identical to SEQ ID NO: 75
  REGION 1..119: note = Description of Artificial Sequence: Synthetic polypeptide

SEQ ID NO: 112 - protein, 112 aa, synthetic construct; identical to SEQ ID NO: 106
  REGION 1..112: note = Description of Artificial Sequence: Synthetic polypeptide

SEQ ID NO: 113 - protein, 119 aa, synthetic construct; identical to SEQ ID NO: 77
  REGION 1..119: note = Description of Artificial Sequence: Synthetic polypeptide

SEQ ID NO: 114 - protein, 112 aa, synthetic construct; identical to SEQ ID NO: 106
  REGION 1..112: note = Description of Artificial Sequence: Synthetic polypeptide

SEQ ID NO: 115 - protein, 119 aa, synthetic construct; identical to SEQ ID NO: 79
  REGION 1..119: note = Description of Artificial Sequence: Synthetic polypeptide

SEQ ID NO: 116 - protein, 112 aa, synthetic construct; identical to SEQ ID NO: 106
  REGION 1..112: note = Description of Artificial Sequence: Synthetic polypeptide

SEQ ID NO: 117 - protein, 449 aa, synthetic construct
  REGION 1..449: note = Description of Artificial Sequence: Synthetic polypeptide
EVQLQQSGAELVNPGASVKLSCTTSGFNIKDTYIHWVKQRPEQGLEWIGRNDPANGNTKYDPKFQGKATITADTSSSTAYLQLSSLTSEDTAVYYCASYEGYYERFGYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 118 - protein, 219 aa, synthetic construct
  REGION 1..219: note = Description of Artificial Sequence: Synthetic polypeptide
DVVMTQAPLTLSVTIGQPASISCKSSQSLLDRDGKTYLNWLLQRPGQSPKRLIYLVSKLDSGVPDRFTGSGSGTDFTLKISRVEAEDLGVYYCWQDTHFPYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 449 aa, synthetic construct
  REGION 1..449: note = Description of Artificial Sequence: Synthetic polypeptide
EVQLQQSGAELVKPGASVKLSCTASGVNIKDTYMYWVKQRPEQGLEWIGRIDPANGYTRYDPKFQGKATITADTSSNTAHLQLSSLTSEDAAVYYCAVYDGYYESFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 120 - protein, 219 aa, synthetic construct
  REGION 1..219: note = Description of Artificial Sequence: Synthetic polypeptide
DVVMTQAPLTLSVTIGQPASISCKSSQSLLDSDGKTYLNWLLQRPGQSPKRLIYLVSKLDSGVPDRFTGSGSGTDFTLKINRVEAEDLGVYYCWQGTLFPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYSLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 122 - protein, 289 aa, Macaca mulatta
MKTLPAMLGSGRLFWVVFLIPYLDIWNIHGKESCDVQLYIKRQSYHSIFAGDPFKLECPVKYCAHRPQVTWCKLNGTTCVKLEGRHTSWKQEKNLSFFILHFEPVLPSDNGSYRCSANFLSAIIESHSTTLYVTDVKSASERPSKDEMASRPWLLYSLLPLGGLPLLITTCFCLFCFLRRHQGKQNELSDTTGREITLVDVPFKSEQTEASTRQNSQVLLSETGIYDNEPDFCFRMQEGSEVYSNPCLEENKPGIIYASLNHSIIALNSRQARNVKEAPTEYASICVRS

SEQ ID NO: 123 - protein, 306 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYTLLPLGALLLLLACVCLLCFLKRIQGKEKKPSDLAGRDTNLVDIPASSRTNHQALPSGTGIYDNDPWSSMQDESELTISLQSERNNQGIVYASLNHCVIGRNPRQENNMQEAPTEYASICVRS

(19) United States
(12) Patent Application Publication
(10) Pub. No.: US 2025/0388673 A1
(43) Pub. Date: Dec. 25, 2025

(54) ANTIBODY AGAINST BTLA AND USES THEREOF

(71) Applicant: HIFIBIO, INC., Cambridge, MA (US)

(72) Inventors: Juying Li, Cambridge, MA (US); Qian Zhang, Cambridge, MA (US); Bingqing Shen, Hangzhou, Zhejiang (CN); Francisco Adrian, Cambridge, MA (US); Liang Schweizer, Cambridge, MA (US)

(21) Appl. No.: 18/833,730

(22) PCT Filed: Jan. 29, 2023
(86) PCT No.: PCT/CN2023/073699

§ 371 Date: Jul. 26, 2024

(30) Foreign Application Priority Data
Jan. 29, 2022 (WO) PCT/CN2022/075109

(51) Int. Cl. C07K 16/28; A61K 39/00; A61P 37/06

(52) U.S. Cl. CPC C07K 16/2818; A61P 37/06; A61K 2039/505; C07K 2317/24; C07K 2317/33; C07K 2317/76

(57) ABSTRACT

The invention provides monoclonal antibodies and antigen-binding fragments thereof specific for BTLA, and methods of using the same to treat autoimmune disorder, including combination therapy.

### CROSS REFERENCE TO RELATED APPLICATIONS

The instant application is a U.S. National Stage Application filed under 35 U.S.C. § 371, based on International Patent Application No. PCT/CN2023/073699, filed on Jan. 29, 2023, which claims priority to and the benefit of the filing date of PCT/CN2022/075109, filed on Jan. 29, 2022. The entire contents of which, including any drawings and sequence listing, are incorporated herein by reference.

### SEQUENCE LISTING

The instant application contains a Sequence Listing XML file which has been submitted electronically in XML format and is hereby incorporated by reference in its entirety. Said XML copy, created on Jan. 23, 2023, is named 131206-01219_SL_XML.xml and is 170,638 bytes in size.

### BACKGROUND OF THE INVENTION

B and T lymphocyte attenuator (BTLA; also known as CD272) is an inhibitory member of the CD28 family of receptors that also includes CD28, CTLA-4, ICOS, and PD-1 (Watanabe et al., Nat Immunol. 4:670-679, 2003). BTLA is widely expressed throughout the immune system on both myeloid and lymphoid cells (Han et al., J Immunol. 172:5931-9, 2004). Following engagement by its ligand herpesvirus entry mediator (HVEM), BTLA recruits the phosphatases SHP-1 and SHP-2 to its cytoplasmic domain (Sedy et al., Nat Immunol. 6:90-8, 2005), which in turn inhibit the signaling cascades of activating receptors.

BTLA knock-out mice lacking an intact BTLA gene show hyperproliferative B and T cell responses in vitro, higher titers to DNP-KLH post-immunization and an increased sensitivity to EAE (Watanabe et al., Nat. Immunol. 4:670-679, 2003). If observed until old age, BTLA knock-out mice spontaneously develop autoantibodies, an auto immune hepatitis like disease, and inflammatory cell infiltrates in multiple organs (Oya et al., Arthritis Rheum. 58: 2498-2510, 2008). Thus, it appears that the BTLA inhibitory receptor plays a crucial role in maintaining immune homeostasis and inhibiting autoimmunity. Furthermore, HVEM-BTLA signaling is involved in the regulation of mucosal inflammation and infection immunity (Shui et al., J Leukoc Biol. 89:517-523, 2011).

Monoclonal antibodies binding to mouse BTLA can act as agonists, inducing signaling through the receptor to inhibit immune cell responses. In the presence of agonist anti-BTLA antibody (mAh), anti-CD3 and anti-CD28 activated T-cells show reduced IL-2 production and proliferation (Kreig et al., J. Immunol. 175, 6420-6472, 2005). Anti-mouse-BTLA agonist antibodies have been shown to ameliorate disease in murine models of graft-versus-host disease (Sakoda et al., Blood 117:2506-2514; Albring et al., J Exp Med. 207:2551-9, 2010). BTLA Agonist antibodies targeting the human BTLA receptor have been shown to inhibit T cell responses ex vivo (Otsuki et al., Biochem Biophys Res Commun 344:1121-7, 2006; and WO2011/014438), yet not such antibodies has been approved to treat any diseases.

Thus, there is a need to develop therapeutic reagents that are capable of modulating BTLA function to inhibit autoreactive lymphocytes in the context of autoimmune disorders.

### SUMMARY OF THE INVENTION

One aspect of the invention provides an isolated monoclonal antibody, or an antigen-binding fragment thereof, wherein said monoclonal antibody or antigen-binding fragment thereof is specific for and activates human BTLA (B- and T-Lymphocyte Attenuator) and optionally cross-reactive with cynomolgus BTLA, and wherein said monoclonal antibody comprises: (1a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and, (1b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6; or, (2a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and, (2b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66; optionally, said monoclonal antibody is not naturally occurring.

In certain embodiments, in the isolated monoclonal antibody or antigen-binding fragment thereof of the invention, (1A) the HCVR sequence is SEQ ID NO: (7+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and, (1B) the LCVR sequence is SEQ ID NO: (8+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region, wherein n is any one of 1, 0, and 2-24; or, (2A) the HCVR sequence is SEQ ID NO: (67+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and, (2B) the LCVR sequence is SEQ ID NO: (68+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region, wherein n is any one of 0-24.

In certain embodiments, the isolated monoclonal antibody or antigen-binding fragment thereof is an IgG1 antibody, an IgG2 antibody, an IgG3 antibody, or an IgG4 antibody.

In certain embodiments, the isolated monoclonal antibody is an IgG1 antibody.

In certain embodiments, the IgG1 antibody comprises a heavy chain constant region sequence of SEQ ID NO: 57 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein, and a light chain constant region sequence of SEQ ID NO: 58 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein.

In certain embodiments, the monoclonal antibody is an IgG1 antibody comprising a mutation in the heavy chain constant region that: (1) modulates antibody dependent cell mediated cytotoxicity (ADCC) and/or antibody dependent cell mediated phagocytosis (ADCP) (such as F243L, G236A, S239D/I332E, S239D/A330L/I332E, S298A/E333A/K334A, F243L/R292P/Y300L/V305I/P396L, or afucosylated (non-fucosylated) antibody (such as afucosylated N297 at Fc region) with enhanced ADCC through increased binding to FcγRIIIa); and/or, (2) enhances serum half-life (such as T250Q/M428L, M252Y/S254T/T256E).

In certain embodiments, the isolated monoclonal antibody or antigen-binding fragment thereof is a mouse antibody, a human-mouse chimeric antibody, a humanized antibody, a human antibody, a CDR-grafted antibody, or a resurfaced antibody.

In certain embodiments, the isolated monoclonal antibody or antigen-binding fragment thereof is a humanized antibody.

In certain embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the isolated monoclonal antibody or antigen-binding fragment thereof cross-reacts with cynomolgus/rhesus monkey BTLA, but does not substantially cross-react with mouse BTLA.

In certain embodiments, the isolated monoclonal antibody or antigen-binding fragment thereof binds human BTLA with a K_{D} of less than about 25 nM, 20 nM, 15 nM, 10 nM, 5 nM, 2 nM, 1 nM, 0.5 nM or less.

In certain embodiments, the isolated monoclonal antibody or antigen-binding fragment thereof is an agonist of human BTLA and activates downstream signaling from BTLA upon binding to BTLA. For example, in certain embodiments, the downstream signaling from BTLA inhibits B cell proliferation, and/or inhibits T cell (e.g., CD4, CD8, Th1, TFH, αβ, or γδ T-cell) and/or plasma cell activation.

In certain embodiments, the isolated monoclonal antibody or antigen-binding fragment thereof does not block/interfere/abolish BTLA binding to HVEM.

In certain embodiments, the isolated monoclonal antibody or antigen-binding fragment thereof does not lead to significant weight loss when administered (e.g., i.p. to mouse) at a dose of 10 mg/kg BIW*6 (e.g., for 21 days).

In certain embodiments, the isolated monoclonal antibody or antigen-binding fragment thereof significantly reduces CD45⁺ T cells when administered in vivo (e.g., i.p. to mouse at a dose of 10 mg/kg BIW*6 for 14 days).

In certain embodiments, the isolated monoclonal antibody or antigen-binding fragment thereof significantly reduces disease secerity (e.g., mean total disease score of less than 3 at 3 weeks) and/or overall survival rate (e.g., overall survival rate of at least 80% at 4 weeks) in GvHD or an animal model thereof, compared to isotype matched control Ab, e.g., when the monoclonal antibody or antigen-binding fragment thereof is administered in vivo (e.g., i.p. to mouse at a dose of 10 mg/kg BIW*6 for 14 days).

Another aspect of the invention provides an isolated monoclonal antibody or an antigen-binding fragment thereof, which competes with the isolated monoclonal antibody or antigen-binding fragment thereof of the invention for binding to human BTLA.

In certain embodiments, the isolated monoclonal antibody or antigen-binding fragment thereof inhibits B cell proliferation, and/or inhibits T cell (e.g., CD4, CD8, Th1, TFH, αβ, or γδ T-cell) activation, upon binding to BTLA.

In certain embodiments, the isolated monoclonal antibody or antigen-binding fragment thereof of the invention comprises: (1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or, (2) the heavy chain amino acid sequence of SEQ ID NO: 59, and the light chain amino acid sequence of SEQ ID NO: 60.

Another aspect of the invention provides an isolated monoclonal antibody or antigen-binding fragment thereof, comprising/consisting essentially of/consisting of: (1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or, (2) the heavy chain amino acid sequence of SEQ ID NO: 59, and the light chain amino acid sequence of SEQ ID NO: 60.

Another aspect of the invention provides a polynucleotide encoding the heavy chain or the light chain or the antigen-binding portion thereof of the invention.

In certain embodiments, the polynucleotide of the invention is codon optimized for expression in a human cell.

Another aspect of the invention provides a vector comprising the polynucleotide of the invention.

In certain embodiments, the vector is an expression vector (e.g., a mammalian, yeast, insect, or bacterial expression vector).

Another aspect of the invention provides a pharmaceutical composition comprising the isolated monoclonal antibody or antigen-binding fragment thereof of the invention, the polynucleotide of the invention, or the vector of the invention.

In certain embodiments, the pharmaceutical composition is formulated for intravenous (i.v.) infusion or administration, or for subcutaneous (s.c.) administration.

Another aspect of the invention provides a method of down-regulating B-cell-mediated or T-cell-mediated immune response, or treating an autoimmune disorder, in a patient in need thereof, the method comprising administering to the patient an effective amount of the isolated monoclonal antibody or antigen-binding fragment thereof of the invention, the polynucleotide of the invention, the vector of the invention, or the pharmaceutical composition of the invention.

In certain embodiments, the method is for treating an autoimmune disorder.

In certain embodiments, the autoimmune disorder is Systemic Lupus Erythematosus (SLE); ulcerative colitis (UC) including Pediatric Ulcerative Colitis; rheumatoid arthritis (RA); psoriasis (Ps) including Chronic Plaque Psoriasis; psoriac arthritis (PsA); Crohn's Disease (CD) including Pediatric Crohn's Disease; Inflammatory Bowel Disease (IBD); ankylosing spondylitis; Juvenile Idiopathic Arthritis (JIA) including Polyarticular Juvenile Idiopathic Arthritis; Hidradenitis Suppurativa; Non-Infectious Intermediate, Posterior, and Panuveitis; autoimmune hepatitis-like diseases; experimental autoimmune encephalomyelitis (EAE); MHC-mismatched cardiac allograft; inflammation of the lung in acute airway allergy; graft versus host disease (GvHD); or allogeneic hematopoietic stem cell transplantation (aHSCT).

In certain embodiments, the method further comprises administering to the patient a further agent effective to treat the autoimmune disorder.

Another aspect of the invention provides a use of the isolated monoclonal antibody or antigen-binding fragment thereof of the invention, the polynucleotide of the invention, the vector of the invention, or the pharmaceutical composition of the invention, in the manufacture of a medicament for down-regulating B-cell-mediated or T-cell-mediated immune response, or for treating an autoimmune disorder, in a patient in need thereof.

Another aspect of the invention provides a composition comprising the isolated monoclonal antibody or antigen-binding fragment thereof of the invention, the polynucleotide of the invention, the vector of the invention, or the pharmaceutical composition of the invention, for use in down-regulating B-cell-mediated or T-cell-mediated immune response, or for treating an autoimmune disorder.

It should be understood that any one embodiment of the invention described herein, including those described only in the examples or claims, can be combined with any other one or more embodiments of the invention, except that the combination is expressly disclaimed or is improper.

### BRIEF DESCRIPTION OF THE DRAWINGS

FIG. 1 shows binding of anti-BTLA antibodies to HEK293 cells overexpressing human BTLA (HEK293.hBTLA) or Expi293 cells overexpressing cynomolgus BTLA (HEK293.cynoBTLA). Anti-BTLA antibody binding to target cells were quantified with an AF647-conjugated anti-human IgG Fc secondary antibody by flow cytometry (readout: mean fluorescence intensity, MFI).

FIG. 2A shows inhibition of primary B cell proliferation 4 days post-anti-IgM stimulation by control and candidate anti-BTLA antibodies. HFB6-4 and HFB6-5 were selected based on their ability to inhibit anti-IgM mediated B cell proliferation in a similar or greater extent than benchmark 22B3. B cell proliferation was quantified by CFSE dilution (% CFSElo) related to nonstimulated B cells and showed as % divided.

FIG. 2B shows inability of HFB6-4 and HFB6-5 to displace HVEM, a natural ligand of HVEM, when bound to BTLA. The top panel is an illustrative scheme showing a CHO cell line enginnered to express a TCR activator (TCRa) and HVEM (the ligand of BTLA), co-cultured with a Jurkat reporter cell line that expresses BTLA and a luciferase reporter driven by NFAT binding promoter. Positive control anti-BTLA blocking antibody JS004, and many other tested anti-BTLA antibodies (see bottom panel), could reverse HVEM-mediated T cell inhibition in a dose-dependent manner. But the ability of HFB6-4 and HFB-5 to reverse HVEM-mediated T cell inhibition was substantially abolished (see bottom panel).

FIG. 3 shows inhibition of TCR signaling by HFB6-4 and HFB6-5 compared to a benchmark antibody 22B3. The left panel depicts the assay: CHO cells expressing FcγRIIb on the cell surface were first incubated with an anti-BTLA antibody, isotype control MGO or benchmark antibody 22B3, with or without an anti-CD3 antibody. The antibody-covered CHO cells were then incubated with a Jurkat reporter cell expressing surface TCR and BTLA. TCR signaling was measured in the reporter cell via activity of a luciferase reporter gene under the transcriptional control of a NFAT promoter downstream of TCR signaling. The right panel shows luminescence readouts from testing different antibodies in the assay.

FIG. 4 shows inhibition of primary B cell proliferation after anti-IgM stimulation (10 μg/ml) by isotype control (MGO53), benchmark antibody 22B3, HFB6-4 and HFB6-5 antibodies. The effects of the antibodies were tested on primary B cells from 3 different donors (left panel) and at varying concentrations (0.41-100 nM) (right panel).

FIG. 5 shows binding of humanized variants of HFB6-4 and HFB6-5 antibodies to HEK293 cells overexpressing human BTLA (HEK293.hBTLA) or Expi293 cells overexpressing cynomolgus BTLA (HEK293.cynoBTLA). Anti-BTLA antibody binding to target cells were quantified with an AF647-conjugated anti-human IgG Fc secondary antibody by flow cytometry (readout: mean fluorescence intensity, MFI).

FIG. 6A shows inhibition of anti-IgM induced primary B cell proliferation by isotype control antibody (MGO53), benchmark antibody 22B3 and humanized variants of HFB6-4 and HFB6-5 at 1 and 10 μg/ml concentrations. B cell proliferation was quantified by CFSE dilution (% CFSElo) related to nonstimulated B cells and showed as % divided.

FIG. 6B shows inhibition of anti-CD3 induced Jurkat cell activation by isotype control antibody (MGO53), benchmark antibody 22B3 and humanized variants of HFB6-4 and HFB6-5, measured by luminescent signal produced by activity of the luminescent reporter gene in the Jurkat reporter cell (RLU: relative light unit).

FIG. 7 shows pharmacokinetic profiles of humanized variant of HFB6-4 and HFB6-5 in C57BL6 mice after intravenous injection of a 10 mg/kg dose. Blood was sampled at 1, 24 and 72 hours post injection.

FIGS. 8A-8G show therapeutic efficacy of the antibodies of interest in inhibiting immune reaction after inoculation of human peripheral blood mononuclear cells (hPBMCs) in a NSG mouse model of acute graft versus host disease (GvHD). FIG. 8A shows a schematic of the experiment. FIG. 8B shows detailed experimental design and readouts for the experiment. FIGS. 8C-8G show preliminary results up to 14-21 days post inoculation, including total disease score (FIG. 8C), percent of RCBW (FIG. 8D), percentage of hCD45+ cells under lymphocyte population (FIG. 8E) and percentage of survival (FIGS. 8F and 8G).

### DETAILED DESCRIPTION OF THE INVENTION

### 1. Overview

The invention described herein provides isolated monoclonal antibodies, or antigen-binding fragments thereof, that are specific for and activates human BTLA (B- and T-Lymphocyte Attenuator), i.e., BTLA agonist antibodies. The BTLA agonist antibodies of the invention optionally cross-react with non-human primate BTLA, such as monkey (e.g., rhesus monkey and/or cynomolgus monkey) BTLA, but may not cross-react with rodent (e.g., mouse or rat BTLA).

According to the invention described herein, certain BTLA agonist antibodies of the invention were initially obtained by immunizing mice with the recombinant extracellular domain (ECD) of human BTLA to produce a series of diverse antibodies that were subsequently characterized for their binding, cross-reactivity, selectivity and/or other functional activities. Certain such antibodies were then selected for their ability to inhibit IgM-induced proliferation of primary B cells and/or T-cells. The functionally selected antibodies may also exhibit cross-reactivity against non-human primate (such as monkey) orthologs of human BTLA, which could be a beneficial feature for toxicity study of a human therapeutic agents in non-human host (e.g., non-human primate or NHP) animals.

Two exemplary mouse antibodies, designated herein as HFB6-4 and HFB6-5, with sub- or single-digit-nanomolar binding affinities towards human BTLA, were initially identified for further characterization and humanization.

Several humanized variants of these mouse antibodies, including HFB6-4hz1-hG1, were produced as with IgG1 isotype, and retained the binding and cross-reactivity profiles of their respective mouse-human chimeric parental antibodies, as well as the inhibitory effects on immune cells, including B-cells, T-cells, and/or plasma cells.

Preliminary in vitro and in vivo efficacy evaluations for these antibodies in mouse immune-disorder models were conducted, so were initial toxicity and developability analysis. Preliminary data shows that the subject monoclonal antibodies are more effective than certain benchmark antibodies as BTLA agonists, and inhibits B- and T-cells more effectively.

The functional profile of these antibodies along with their favorable developability and pharmacokinetic profiles support their development as novel therapeutic agents for treating immune disorders, such as various autoimmune diseases (e.g., graft versus host disease or GvHD).

Thus one aspect of the invention provides an isolated monoclonal antibody, or an antigen-binding fragment thereof, wherein the monoclonal antibody or antigen-binding fragment thereof is specific for and activates human BTLA (B- and T-Lymphocyte Attenuator), and optionally cross-reactive with cynomolgus BTLA, and wherein said monoclonal antibody comprises: (1a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and, (1b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6; OR, (2a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and, (2b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66; optionally, said monoclonal antibody is not naturally occurring.

In certain embodiments, the CDR region sequences are based on IMGT numbering scheme. CDR sequences corresponding to other numbering schemes such as Kabat, Chothia, or Martin (enhanced Chothia) schemes can be readily derived by antibody sequence alignments.

In a related aspect, the invention provides an isolated monoclonal antibody or antigen-binding fragment thereof, comprising/consisting essentially of/consisting of: (1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; OR, (2) the heavy chain amino acid sequence of SEQ ID NO: 59, and the light chain amino acid sequence of SEQ ID NO: 60.

In a related aspect, the invention provides an isolated monoclonal antibody or an antigen-binding fragment thereof, which competes with the isolated monoclonal antibody or antigen-binding fragment thereof having the defined CDR and/or VH/VL region sequences as defined here, for binding to human BTLA (such as SEQ ID NO: 121).

Another aspect of the invention provides a polynucleotide encoding the heavy chain or the light chain or the antigen-binding portion thereof of any of the moniclonal antibody or antigen-binding fragment thereof as described herein. In a related aspect, the invention also provides a polynucleotide that hybridizes under stringent conditions to the polynucleotide, or with a complement of the polynucleotide.

Another aspect of the invention provides a vector comprising the polynucleotide of the invention as described herein.

Another aspect of the invention provides a pharmaceutical composition comprising the isolated monoclonal antibody or antigen-binding fragment thereof of the invention, the polynucleotide of the invention, or the vector of the invention.

Another aspect of the invention provides a method of down-regulating B-cell-mediated or T-cell-mediated immune response, or for treating an autoimmune disorder, in a patient in need thereof, the method comprising administering to the patient an effective amount of the isolated monoclonal antibody or antigen-binding fragment thereof of the invention, the polynucleotide of the invention, the vector of the invention, or the pharmaceutical composition of the invention.

Another aspect of the invention provides a use of the isolated monoclonal antibody or antigen-binding fragment thereof of the invention, the polynucleotide of the invention, the vector of the invention, or the pharmaceutical composition of the invention, in the manufacture of a medicament for down-regulating B-cell-mediated or T-cell-mediated immune response, or for treating an autoimmune disorder, in a patient in need thereof.

Another aspect of the invention provides a composition comprising the isolated monoclonal antibody or antigen-binding fragment thereof of the invention, the polynucleotide of the invention, the vector of the invention, or the pharmaceutical composition of the invention, for use in down-regulating B-cell-mediated or T-cell-mediated immune response, or for treating an autoimmune disorder.

Detailed aspects of the invention are described further and separately in the various sections below. However, it should be understood that any one embodiment of the invention, including embodiments described only in the examples or drawings, and embodiments described only under one section below, can be combined with any other embodiment(s) of the invention.

### 2. Definitions

The term “antibody,” in the broadest sense, encompasses various antibody structures, including but not limited to monoclonal antibodies, polyclonal antibodies, and multispecific antibodies (e.g., bispecific antibodies). The term “antibody” may also broadly refers to a molecule comprising complementarity determining region (CDR) 1, CDR2, and CDR3 of a heavy chain and CDR1, CDR2, and CDR3 of a light chain, wherein the molecule is capable of binding to an antigen. The term “antibody” also includes, but is not limited to, chimeric antibodies, humanized antibodies, human antibodies, and antibodies of various species such as mouse, human, cynomolgus monkey, etc.

In a narrower sense, however, “antibody” refers to the various monoclonal antibodies, including chimeric monoclonal antibodies, humanized monoclonal antibodies, and human monoclonal antibodies, particularly humanized monoclonal antibodies of the invention.

In some embodiments, an antibody comprises a heavy chain variable region (HCVR) and a light chain variable region (LCVR). In some embodiments, an antibody comprises at least one heavy chain (HC) comprising a heavy chain variable region and at least a portion of a heavy chain constant region, and at least one light chain (LC) comprising a light chain variable region and at least a portion of a light chain constant region. In some embodiments, an antibody comprises two heavy chains, wherein each heavy chain comprises a heavy chain variable region and at least a portion of a heavy chain constant region, and two light chains, wherein each light chain comprises a light chain variable region and at least a portion of a light chain constant region.

As used herein, a single-chain Fv (scFv), or any other antibody that comprises, for example, a single polypeptide chain comprising all six CDRs (three heavy chain CDRs and three light chain CDRs) is considered to have a heavy chain and a light chain. In some such embodiments, the heavy chain is the region of the antibody that comprises the three heavy chain CDRs and the light chain in the region of the antibody that comprises the three light chain CDRs.

The term “heavy chain variable region (HCVR)” as used herein refers to, at a minimum, a region comprising heavy chain CDR1 (CDR-H1), framework 2 (HFR2), CDR2 (CDR-H2), FR3 (HFR3), and CDR3 (CDR-H3). In some embodiments, a heavy chain variable region also comprises at least a portion (e.g., the whole) of an FR1 (HFR1), which is N-terminal to CDR-H1, and/or at least a portion (e.g., the whole) of an FR4 (HFR4), which is C-terminal to CDR-H3.

The term “heavy chain constant region” as used herein refers to a region comprising at least three heavy chain constant domains, CH1, CH2, and CH3. Non-limiting exemplary heavy chain constant regions include γ, δ, and α. Non-limiting exemplary heavy chain constant regions also include ε and μ. Each heavy constant region corresponds to an antibody isotype. For example, an antibody comprising a γ constant region is an IgG antibody, an antibody comprising a δ constant region is an IgD antibody, an antibody comprising an α constant region is an IgA antibody, an antibody comprising an F constant region is an IgE antibody, and an antibody comprising an μ constant region is an IgM antibody.

Certain isotypes can be further subdivided into subclasses. For example, IgG antibodies include, but are not limited to, IgG1 (comprising a γ1 constant region), IgG2 (comprising a γ2 constant region), IgG3 (comprising a γ3 constant region), and IgG4 (comprising a γ4 constant region) antibodies; IgA antibodies include, but are not limited to, IgA1 (comprising an al constant region) and IgA2 (comprising an α2 constant region) antibodies; and IgM antibodies include, but are not limited to, IgM1 (comprising an 1 constant region) and IgM2 (comprising an μ2 constant region).

In certain embodiments, the antibodies of the invention are IgG1 antibodies.

The term “heavy chain” as used herein refers to a polypeptide comprising at least a heavy chain variable region, with or without a leader sequence. In some embodiments, a heavy chain comprises at least a portion of a heavy chain constant region. The term “full-length heavy chain” as used herein refers to a polypeptide comprising a heavy chain variable region and a heavy chain constant region, with or without a leader sequence, and with or without a C-terminal lysine.

The term “light chain variable region (LCVR)” as used herein refers to a region comprising light chain CDR1 (CDR-L1), framework (FR) 2 (LFR2), CDR2 (CDR-L2), FR3 (LFR3), and CDR3 (CDR-L3). In some embodiments, a light chain variable region also comprises at least a portion (e.g., the whole) of an FR1 (LFR1) and/or at least a portion (e.g., the whole) of an FR4 (LFR4).

The term “light chain constant region” as used herein refers to a region comprising a light chain constant domain, C_{L}. Non-limiting exemplary light chain constant regions include λ and κ.

The term “light chain” as used herein refers to a polypeptide comprising at least a light chain variable region, with or without a leader sequence. In some embodiments, a light chain comprises at least a portion of a light chain constant region. The term “full-length light chain” as used herein refers to a polypeptide comprising a light chain variable region and a light chain constant region, with or without a leader sequence.

The term “antibody fragment,” “antigen binding portion” or “antigen-binding fragment” (of antibody) includes, but is not limited to, fragments that are capable of binding antigen, such as Fv, single-chain Fv (scFv), Fab, Fab′, and (Fab′)₂. In certain embodiments, an antibody fragment includes Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

The term “Fab” refers to an antibody fragment with a molecular mass of approximately 50,000 Daltons, and has an activity of binding to the antigen. It comprises approximately half of the N-terminal side of the heavy chain and the whole of the light chain connected by a disulfide bridge. The Fab can be obtained in particular by treatment of immunoglobulin by a protease, papain.

The term “F(ab′)₂” designates a fragment of approximately 100,000 Daltons and an activity of binding to the antigen. This fragment is slightly larger than two Fab fragments connected via a disulfide bridge in the hinge region. These fragments are obtained by treating an immunoglobulin with a protease, pepsin. The Fab fragment can be obtained from the F(ab′)2 fragment by cleaving of the disulfide bridge of the hinge region. A single Fv chain “scFv” corresponds to a VH: VL polypeptide synthesized using the genes coding for the VL and VH domains and a sequence coding for a peptide intended to bind these domains. An scFv according to the invention includes the CDRs maintained in an appropriate conformation, for example using genetic recombination techniques.

The dimers of “scFv” correspond to two scFv molecules connected together by a peptide bond. This Fv chain is frequently the result of the expression of a fusion gene including the genes coding for VH and VL connected by a linker sequence coding a peptide. The human scFv fragment may include CDR regions that are maintained in an appropriate conformation, preferably by means of the use of genetic recombination techniques.

The “dsFv” fragment is a VH-VL heterodimer stabilized by a disulfide bridge; it may be divalent (dsFV₂). Fragments of divalent Sc(Fv)₂ or multivalent antibodies may form spontaneously by the association of monovalent scFvs or be produced by connecting scFvs fragments by peptide binding sequences.

The Fc fragment is the support for the biological properties of the antibody, in particular its ability to be recognized by immunity effectors or to activate the complement. It consists of constant fragments of the heavy chains beyond the hinge region.

The term “diabodies” signifies small antibody fragments having two antigen fixing sites. These fragments comprise, in the same VH-VL polypeptide chain, a variable heavy chain domain VH connected to a variable light chain domain VL. Using a binding sequence that is too short to allow the matching of two domains of the same chain, the matching with two complementary domains of another chain necessarily occurs and thus two antigen fixing sites are created.

An “antibody that binds to the same epitope” or “antibody that competes for binding with (another) antibody” refers to an antibody that can be determined by an antibody competition assay. It refers to an antibody that blocks binding of the reference antibody to its antigen in a competition assay by 50% or more, and conversely, the reference antibody blocks binding of the antibody to its antigen in a competition assay by 50% or more. The term “compete” when used in the context of an antibody that compete for the same epitope means competition between antibodies is determined by an assay in which an antibody being tested prevents or inhibits specific binding of a reference antibody to a common antigen.

Numerous types of competitive binding assays can be used, for example: solid phase direct or indirect radioimmunoassay (RIA), solid phase direct or indirect enzyme immunoassay (EIA), sandwich competition assay (see, e.g., Stahli et al., 1983, Methods in Enzymology 9:242-253); solid phase direct biotin-avidin EIA (see, e.g., Kirkland et al., 1986, J. Immunol. 137:3614-3619); solid phase direct labeled assay; solid phase direct labeled sandwich assay (see, e.g., Harlow and Lane, 1988, Antibodies, A Laboratory Manual, Cold Spring Harbor Press); solid phase direct label RIA using I¹²⁵ label (see, e.g., Morel et al., 1988, Molec. Immunol. 25:7-15); solid phase direct biotin-avidin EIA (see, e.g., Cheung, et al., 1990, Virology 176:546-552); and direct labeled RIA (Moldenhauer et al., 1990, Scand. J. Immunol.).

Typically, such an assay involves the use of purified antigen bound to a solid surface or cells bearing either of these, an unlabeled test antigen binding protein and a labeled reference antibody. Competitive inhibition is measured by determining the amount of label bound to the solid surface or cells in the presence of the test antibody. Usually the test antibody is present in excess. Antibodies identified by competition assay (competing antibodies) include antibodies binding to the same epitope as the reference antibodies and antibodies binding to an adjacent epitope sufficiently proximal to the epitope bound by the reference antibody for steric hindrance to occur. In some embodiments, when a competing antibody is present in excess, it will inhibit specific binding of a reference antibody to a common antigen by at least 40%, 45%, 50%, 55%, 60%, 65%, 70% or 75%. In some instance, binding is inhibited by at least 80%, 85%, 90%, 95%, or 97% or more.

The term “antigen” refers to a molecule or a portion of a molecule capable of being bound by a selective binding agent, such as an antibody or immunologically functional fragment thereof, and additionally capable of being used in a mammal to produce antibodies capable of binding to that antigen. An antigen may possess one or more epitopes that are capable of interacting with antibodies.

The term “epitope” is the portion of an antigen molecule that is bound by a selective binding agent, such as an antibody or a fragment thereof. The term includes any determinant capable of specifically binding to an antibody. An epitope can be contiguous or non-contiguous (e.g., in a polypeptide, amino acid residues that are not contiguous to one another in the polypeptide sequence but that within in context of the molecule are bound by the antigen binding protein). In some embodiments, epitopes may be mimetic in that they comprise a three dimensional structure that is similar to an epitope used to generate the antibody, yet comprise none or only some of the amino acid residues found in that epitope used to generate the antibody. Epitope determinants may include chemically active surface groupings of molecules such as amino acids, sugar side chains, phosphoryl or sulfonyl groups, and may have specific three dimensional structural characteristics, and/or specific charge characteristics.

In some embodiments, an “epitope” is defined by the method used to determine it. For example, in some embodiments, an antibody binds to the same epitope as a reference antibody, if they bind to the same region of the antigen, as determined by hydrogen-deuterium exchange (HDX).

In certain embodiments, an antibody binds to the same epitope as a reference antibody if they bind to the same region of the antigen, as determined by X-ray crystallography.

A “chimeric antibody” as used herein refers to an antibody comprising at least one variable region from a first species (such as mouse, rat, cynomolgus monkey, etc.) and at least one constant region from a second species (such as human, cynomolgus monkey, chicken, etc.). In some embodiments, a chimeric antibody comprises at least one mouse variable region and at least one human constant region. In some embodiments, all of the variable regions of a chimeric antibody are from a first species and all of the constant regions of the chimeric antibody are from a second species.

A “humanized antibody” as used herein refers to an antibody in which at least one amino acid in a framework region of a non-human variable region (such as mouse, rat, cynomolgus monkey, chicken, etc.) has been replaced with the corresponding amino acid from a human variable region. In some embodiments, a humanized antibody comprises at least one human constant region or fragment thereof. In some embodiments, a humanized antibody fragment is an Fab, an scFv, a (Fab′)₂, etc.

A “CDR-grafted antibody” as used herein refers to a humanized antibody in which one or more complementarity determining regions (CDRs) of a first (non-human) species have been grafted onto the framework regions (FRs) of a second (human) species.

A “human antibody” as used herein refers to antibodies produced in humans, antibodies produced in non-human animals that comprise human immunoglobulin genes, such as XENOMOUSE®, and antibodies selected using in vitro methods, such as phage display, wherein the antibody repertoire is based on a human immunoglobulin sequences.

A “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells; fungal cells, such as yeast; plant cells; and insect cells. Non-limiting exemplary mammalian cells include, but are not limited to, NSO cells, PER.C6® cells (Crucell), and 293 and CHO cells, and their derivatives, such as 293-6E and DG44 cells, respectively.

The term “isolated” as used herein refers to a molecule that has been separated from at least some of the components with which it is typically found in nature or has been separated from at least some of the components with which it is typically produced. For example, a polypeptide is referred to as “isolated” when it is separated from at least some of the components of the cell in which it was produced. Where a polypeptide is secreted by a cell after expression, physically separating the supernatant containing the polypeptide from the cell that produced it is considered to be “isolating” the polypeptide. Similarly, a polynucleotide is referred to as “isolated” when it is not part of the larger polynucleotide (such as, for example, genomic DNA or mitochondrial DNA, in the case of a DNA polynucleotide) in which it is typically found in nature, or is separated from at least some of the components of the cell in which it was produced, e.g., in the case of an RNA polynucleotide. Thus, a DNA polynucleotide that is contained in a vector inside a host cell may be referred to as “isolated” so long as that polynucleotide is not found in that vector in nature.

The terms “subject” and “patient” are used interchangeably herein to refer to a mammal such as human. In some embodiments, methods of treating other non-human mammals, including, but not limited to, rodents, simians, felines, canines, equines, bovines, porcines, ovines, caprines, mammalian laboratory animals, mammalian farm animals, mammalian sport animals, and mammalian pets, are also provided. In some instances, a “subject” or “patient” refers to a (human) subject or patient in need of treatment for a disease or disorder.

The term “sample” or “patient sample” as used herein, refers to material that is obtained or derived from a subject of interest that contains a cellular and/or other molecular entity that is to be characterized and/or identified, for example based on physical, biochemical, chemical and/or physiological characteristics. For example, the phrase “disease sample” and variations thereof refers to any sample obtained from a subject of interest that would be expected or is known to contain the cellular and/or molecular entity that is to be characterized.

By “tissue or cell sample” is meant a collection of similar cells obtained from a tissue of a subject or patient. The source of the tissue or cell sample may be solid tissue as from a fresh, frozen and/or preserved organ or tissue sample or biopsy or aspirate; blood or any blood constituents; bodily fluids such as sputum, cerebral spinal fluid, amniotic fluid, peritoneal fluid, or interstitial fluid; cells from any time in gestation or development of the subject. The tissue sample may also be primary or cultured cells or cell lines. Optionally, the tissue or cell sample is obtained from a disease tissue/organ. The tissue sample may contain compounds which are not naturally intermixed with the tissue in nature such as preservatives, anticoagulants, buffers, fixatives, nutrients, antibiotics, or the like.

A “reference sample,” “reference cell,” or “reference tissue,” as used herein, refers to a sample, cell or tissue obtained from a source known, or believed, not to be afflicted with the disease or condition for which a method or composition of the invention is being used to identify. In one embodiment, a reference sample, reference cell or reference tissue is obtained from a healthy part of the body of the same subject or patient in whom a disease or condition is being identified using a composition or method of the invention. In one embodiment, a reference sample, reference cell or reference tissue is obtained from a healthy part of the body of at least one individual who is not the subject or patient in whom a disease or condition is being identified using a composition or method of the invention. In some embodiments, a reference sample, reference cell or reference tissue was previously obtained from a patient prior to developing a disease or condition or at an earlier stage of the disease or condition.

A “disorder” or “disease” is any condition that would benefit from treatment with one or more BTLA angonists of the invention. This includes chronic and acute disorders or diseases including those pathological conditions that predispose the mammal to the disorder in question. Non-limiting examples of disorders to be treated herein include autoimmune disorders.

An “overactive immune disorder” means any illnesses in which the immune system undesirably attacks a cell, an organ or a graft in a subject, or in which cells of the immune system proliferates uncontrollably. Common overactive immune disorders include autoimmune disorders and rejection of allogenic transplants such as graft versus host disease.

“Treatment” refers to therapeutic treatment, for example, wherein the object is to slow down (lessen) the targeted pathologic condition or disorder as well as, for example, wherein the object is to inhibit recurrence of the condition or disorder. “Treatment” covers any administration or application of a therapeutic for a disease (also referred to herein as a “disorder” or a “condition”) in a mammal, including a human, and includes inhibiting the disease or progression of the disease, inhibiting or slowing the disease or its progression, arresting its development, partially or fully relieving the disease, partially or fully relieving one or more symptoms of a disease, or restoring or repairing a lost, missing, or defective function; or stimulating an inefficient process. The term “treatment” also includes reducing the severity of any phenotypic characteristic and/or reducing the incidence, degree, or likelihood of that characteristic. Those in need of treatment include those already with the disorder as well as those at risk of recurrence of the disorder or those in whom a recurrence of the disorder is to be prevented or slowed down.

The term “effective amount” or “therapeutically effective amount” refers to an amount of a drug effective to treat a disease or disorder in a subject. In some embodiments, an effective amount refers to an amount effective, at dosages and for periods of time necessary, to achieve the desired therapeutic or prophylactic result. A therapeutically effective amount of the antibodies of the invention may vary according to factors such as the disease state, age, sex, and weight of the individual, and the ability of the antagonist to elicit a desired response in the individual. A therapeutically effective amount encompasses an amount in which any toxic or detrimental effects of the subject antibodies are outweighed by the therapeutically beneficial effects.

A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

A “pharmaceutically acceptable carrier” refers to a non-toxic solid, semisolid, or liquid filler, diluent, encapsulating material, formulation auxiliary, or carrier conventional in the art for use with a therapeutic agent that together comprise a “pharmaceutical composition” for administration to a subject. A pharmaceutically acceptable carrier is non-toxic to recipients at the dosages and concentrations employed and is compatible with other ingredients of the formulation. The pharmaceutically acceptable carrier is appropriate for the formulation employed. For example, if the therapeutic agent is to be administered orally, the carrier may be a gel capsule. If the therapeutic agent is to be administered subcutaneously, the carrier ideally is not irritable to the skin and does not cause injection site reaction.

An “article of manufacture” is any manufacture (e.g., a package or container) or kit comprising at least one reagent, e.g., a medicament for treatment of a disease or disorder, or a probe for specifically detecting a biomarker described herein. In some embodiments, the manufacture or kit is promoted, distributed, or sold as a unit for performing the methods described herein.

### 3. Methods of Treating Overactive Immune Disorders

The invention described herein provides anti-BTLA antibodies for use in methods of treating humans and other non-human mammals.

In a pathological situation, the immune system may be overactive and attack normal and healthy tissues, leading to autoimmune diseases. In other pathological situations, cells of the immune system may be overproliferative, e.g., a lymphoma (such as B-cell lymphoma). In other situations, the immune system attacks foreign entity as intended, but acceptance of an allogenic entity is the desired outcome, such as in the case of an allogenic transplantation.

Therefore, in some non-liming embodiments, the anti-BTLA agonist antibodies of the invention or antigen-binding fragments thereof can be used for a subject that will benefit from a treatment that dampens the immune response, e.g., a subject having an autoimmune disease or a subject receiving an allogenic graft. Anti-BTLA antibodies of the invention result in suppression of the immune response through, for example, inhibiting proliferation and/or activity of T-cells and/or B cells and plasma cells.

That is, the invention described herein provides a method of down-regulating B-cell-mediated or T-cell-mediated immune response, or treating an autoimmune disorder, in a patient in need thereof, the method comprising administering to the patient an effective amount of the isolated monoclonal antibody or antigen-binding fragment thereof of the invention, the polynucleotide of the invention, the vector of the invention, or the pharmaceutical composition of the invention.

A related aspect provides a use of the isolated monoclonal antibody or antigen-binding fragment thereof of the invention, the polynucleotide of the invention, the vector of the invention, or the pharmaceutical composition of the invention, in the manufacture of a medicament for down-regulating B-cell-mediated or T-cell-mediated immune response, or for treating an autoimmune disorder, in a patient in need thereof.

Another related aspect provides a composition comprising the isolated monoclonal antibody or antigen-binding fragment thereof of the invention, the polynucleotide of the invention, the vector of the invention, or the pharmaceutical composition of the invention, for use in down-regulating B-cell-mediated or T-cell-mediated immune response, or for treating an autoimmune disorder.

In some embodiments, methods for treating or preventing an overactive immune disorder are provided, comprising administering an effective amount of any of the subject anti-BTLA antibodies or antigen-binding fragments thereof to a subject in need of such treatment.

In some embodiments, methods for treating an overactive immune disorder are provided, comprising administering an effective amount of any of the subject anti-BTLA agonist antibodies or antigen-binding fragments thereof to a subject in need thereof, e.g., a subject diagnosed with, having high risk of having, or exhibiting a symptom of having an overactive immune disorder, e.g., autoimmune disease.

Non-limiting exemplary overactive immune disorders, e.g., autoimmune diseases, that may be treated with the subject anti-BTLA antibodies or antigen-binding fragments thereof are provided herein, including Systemic Lupus Erythematosus (SLE); ulcerative colitis (UC) including Pediatric Ulcerative Colitis; rheumatoid arthritis (RA); psoriasis (Ps) including Chronic Plaque Psoriasis; psoriac arthritis (PsA); Crohn's Disease (CD) including Pediatric Crohn's Disease; Inflammatory Bowel Disease (IBD); ankylosing spondylitis; Juvenile Idiopathic Arthritis (JIA) including Polyarticular Juvenile Idiopathic Arthritis; Hidradenitis Suppurativa; Non-Infectious Intermediate, Posterior, and Panuveitis; autoimmune hepatitis-like diseases; experimental autoimmune encephalomyelitis (EAE); MHC-mismatched cardiac allograft; inflammation of the lung in acute airway allergy; graft versus host disease (GvHD); or allogeneic hematopoietic stem cell transplantation (aHSCT).

In certain embodiment, the overactive immune disorder is Systemic Lupus Erythematosus (SLE or Lupus). Lupus is characterized by the pathological formation of pathogenic autoantibodies against nuclear, cytoplasmic, and/or cell surface molecules, resulting from B and T cell immune dysregulation. Local formation and/or deposition of circulating antigen antibody immune complexes trigger inflammatory responses that are responsible for a wide spectrum of systemic and organ-specific clinical presentations, characterized by remissions and exacerbations, leading to multi-organ system damage and, potentially, end-organ failure. Lupus is a multifaceted autoimmune disease characterized by disabling symptoms and progressive organ damage.

In certain embodiment, the overactive immune disorder is Inflammatory Bowel Disease (IBD). IBD encompasses two conditions, Crohn's disease (CD) and ulcerative colitis (UC), that are characterized by chronic inflammation resulting in damage to the gastrointestinal (GI) tract. Some common symptoms of IBD are persistent diarrhea, abdominal pain, rectal bleeding/bloody stools, weight loss and fatigue.

In certain embodiments, the overactive immune disorder is a condition where the immune system of a subject attacks an allogenic graft obtained from a donor, e.g., graft versus host disease (GvHD). GvHD can occur any time after a transplantation, and can be acute or chronic. In a bone-marrow transplantation, GvHD more commonly occurs after the marrow has started to make cells. Symptoms vary based on how long someone has had the condition, but may include mouth ulcers, abdominal pain, and rash.

In some embodiments, methods for preventing an overactive immune disorder are provided, comprising administering an effective amount of any of the subject anti-BTLA antibodies or antigen-binding fragments thereof prophylactically to a subject at risk of developing symptoms of an overactive immune-related disorder. For example, the subject anti-BTLA antibodies or antigen-binding fragments thereof may be administered to a subject prior to receiving an allogenic transplant.

In some embodiments, the anti-BTLA antibodies or antigen-binding fragments thereof of the invention can be used alone, or alternatively used in combination with any other suitable compound known to be able to treat the disease or indication.

Thus according to a particular embodiment of the invention, an antibody directed against BLTA and inhibiting T and/or B cells and plasma cells, as previously defined, is used in combination with a second therapeutic agent for treating a disease associated with an overactive immune system, e.g., an autoimmune disease such as Lupus.

In such combination therapy, the antibody of the invention can be used before, after, or concurrently with the second therapeutic agent. See further section below concerning combination therapy.

### 4. Routes of Administration and Carriers

In various embodiments, the subject agonist anti-BTLA monoclonal antibodies may be administered subcutaneously or intravenously. For simplicity, “the subject anti-BTLA monoclonal antibodies” refer to mouse-human chimeric anti-BTLA antibody of the invention, as well as the humanized variants thereof.

In some embodiments, the subject anti-BTLA monoclonal antibodies may be administered in vivo by various routes, including, but not limited to, oral, intra-arterial, parenteral, intranasal, intramuscular, intracardiac, intraventricular, intratracheal, buccal, rectal, intraperitoneal, intravenous, and subcutaneous administration, by inhalation, intradermal, topical, transdermal, and intrathecal, or otherwise, e.g., by implantation.

In some embodiments, the subject anti-BTLA monoclonal antibodies may be administered via intravenous (i.v.) or subcutaneous (s.c.) administration.

The subject antibody compositions may be formulated into preparations in solid, semi-solid, liquid, or gaseous forms; including, but not limited to, tablets, capsules, powders, granules, ointments, solutions, suppositories, enemas, injections, inhalants, and aerosols.

In various embodiments, compositions comprising the subject anti-BTLA monoclonal antibodies are provided in formulations with a wide variety of pharmaceutically acceptable carriers (see, e.g., Gennaro, Remington: The Science and Practice of Pharmacy with Facts and Comparisons: Drugfacts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)).

Various pharmaceutically acceptable carriers, which include vehicles, adjuvants, and diluents, are available. Moreover, various pharmaceutically acceptable auxiliary substances, such as pH adjusting and buffering agents, tonicity adjusting agents, stabilizers, wetting agents and the like, are also available. Nonlimiting exemplary carriers include saline, buffered saline, dextrose, water, glycerol, ethanol, and combinations thereof.

In various embodiments, compositions comprising the subject anti-BTLA monoclonal antibodies may be formulated for injection, including subcutaneous administration, by dissolving, suspending, or emulsifying them in an aqueous or nonaqueous solvent, such as vegetable or other oils, synthetic aliphatic acid glycerides, esters of higher aliphatic acids, or propylene glycol; and if desired, with conventional additives such as solubilizers, isotonic agents, suspending agents, emulsifying agents, stabilizers and preservatives.

In various embodiments, the compositions may be formulated for inhalation, for example, using pressurized acceptable propellants such as dichlorodifiuoromethane, propane, nitrogen, and the like.

The compositions may also be formulated, in various embodiments, into sustained release microcapsules, such as with biodegradable or non-biodegradable polymers. A non-limiting exemplary biodegradable formulation includes poly lactic acid-glycolic acid (PLGA) polymer. A non-limiting exemplary non-biodegradable formulation includes a polyglycerin fatty acid ester. Certain methods of making such formulations are described, for example, in EP 1125584 A1.

Pharmaceutical dosage packs comprising one or more containers, each containing one or more doses of the subject anti-BTLA monoclonal antibodies, are also provided. In some embodiments, a unit dosage is provided wherein the unit dosage contains a predetermined amount of a composition comprising the subject anti-BTLA monoclonal antibodies, with or without one or more additional agents. In some embodiments, such a unit dosage is supplied in single-use prefilled syringe for injection. In various embodiments, the composition contained in the unit dosage may comprise saline, sucrose, or the like; a buffer, such as phosphate, or the like; and/or be formulated within a stable and effective pH range. Alternatively, in some embodiments, the composition may be provided as a lyophilized powder that may be reconstituted upon addition of an appropriate liquid, for example, sterile water. In some embodiments, the composition comprises one or more substances that inhibit protein aggregation, including, but not limited to, sucrose and arginine. In some embodiments, a composition of the invention comprises heparin and/or a proteoglycan.

Pharmaceutical compositions are administered in an amount effective for treatment or prophylaxis of the specific indication. The therapeutically effective amount is typically dependent on the weight of the subject being treated, his or her physical or health condition, the extensiveness of the condition to be treated, or the age of the subject being treated.

### 5. Combination Therapy

The subject anti-BTLA monoclonal antibodies of the invention, including functional fragments thereof, may be administered to a subject in need thereof in combination with other biologically active substances or other treatment procedures for the treatment of diseases.

For example, the subject anti-BTLA monoclonal antibodies may be administered alone or with other modes of treatment. They may be provided before, substantially contemporaneous with, or after other modes of treatment, such as immunosuppressive drugs.

For treatment of overactive immune disorder, the subject anti-BTLA monoclonal antibodies may be administered in conjunction with one or more of immunosuppressive agents, such as corticosteroids, Janus kinase inhibitors, calcineurin inhibitors, mTOR inhibitors, IMDH inhibitors and other immunosuppressive agents.

In certain embodiments, the subject anti-BTLA monoclonal antibodies is administered with another treatment, either simultaneously, or consecutively, to a subject, e.g., a subject having an autoimmune disorder. For example, the subject anti-BTLA monoclonal antibodies may be administered with one of more of immunosuppressive agents.

In certain embodiments, a method of treatment of a subject having an overactive immune disorder comprises administering to the subject an anti-BTLA monoclonal antibody of the invention, and one or more immunosuppressive agents, such as a corticosteroid, e.g., steroid.

Immunosuppressive agents that may be administered in combination with the subject anti-BTLA monoclonal antibodies include, but are not limited to: non-steroidal anti-inflammatory drugs (NSAIDs) such as ibuprofen, naproxen, diclofenac, celecoxib, mefenamic acid, etoricoxib, indomethacin and high-dose aspirin; antimalarial drugs such as hydroxychloroquine and chloroquine phosphate; corticosteroids such as prednisone, budesonide and prednisolone; Janus kinase inhibitors such as tofacitinib; calcineurin inhibitors such as cyclosporine and tacrolimus; mTOR inhibitors such as sirolumus and everolimus; and IMDH inhibitors such as azathioprine, leflunomide and mycophenolate.

### 6. Exemplary Anti-BTLA Monoclonal Antibody

The invention described herein provides agonist monoclonal antibodies specific for BTLA, or antigen-binding fragments thereof.

Thus one aspect of the invention provides an isolated monoclonal antibody, or an antigen-binding fragment thereof, which competes with any of the isolated monoclonal antibody or antigen-binding fragment thereof described herein for binding to the epitope bound by HFB6-4 or HFB6-5.

Another related aspect of the invention provides an isolated monoclonal antibody, or an antigen-binding fragment thereof, wherein said monoclonal antibody or antigen-binding fragment thereof is specific for human BTLA, and wherein said monoclonal antibody comprises: (1a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and, (1b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6; or (2a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and, (2b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66.

In certain embodiments, the CDR region sequences described herein are based on IMGT numbering scheme. CDR sequences corresponding to other numbering schemes such as Kabat, Chothia, or Martin (enhanced Chothia) schemes can be readily derived by antibody sequence alignments.

For any of the aspects of the invention described above, in some embodiments, in the isolated monoclonal antibody or antigen-binding fragment thereof: (1A) the HCVR sequence is SEQ ID NO: 7; and/or, (1B) the LCVR sequence is SEQ ID NO: 8, or, (2A) the HCVR sequence is SEQ ID NO: 67; and/or, (2B) the LCVR sequence is SEQ ID NO: 68.

In some embodiments, in the isolated monoclonal antibody or antigen-binding fragment thereof, the HCVR sequence is SEQ ID NO: 7 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 8 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region.

In some embodiments, in the isolated monoclonal antibody or antigen-binding fragment thereof, the HCVR sequence is SEQ ID NO: 9 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 10 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 11 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 12 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 13 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 14 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 15 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 16 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 17 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 18 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 19 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 20 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 21 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 22 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 23 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 24 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 25 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 26 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 27 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 28 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 29 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 30 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 31 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 32 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 33 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 34 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 35 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 36 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 37 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 38 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 39 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 40 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 41 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 42 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 43 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 44 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 45 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 46 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 47 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 48 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 49 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 50 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 51 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 52 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 53 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 54 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 55 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 56 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 67 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 68 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region.

In some embodiments, in the isolated monoclonal antibody or antigen-binding fragment thereof, the HCVR sequence is SEQ ID NO: 69 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 70 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 71 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 72 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 73 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 74 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 75 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 76 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 77 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 78 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 79 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 80 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 81 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 82 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 83 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 84 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 85 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 86 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 87 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 88 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 89 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 90 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 91 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 92 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 93 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 94 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 95 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 96 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 97 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 98 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 99 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 100 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 101 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 102 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 103 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 104 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 105 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 106 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 107 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 108 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 109 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 110 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 111 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 112 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 113 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 114 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, in the isolated monoclonal antibody or antigen-binding fragment thereof, the HCVR sequence is SEQ ID NO: 115 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and/or, the LCVR sequence is SEQ ID NO: 116 or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region.

In some embodiments, the isolated monoclonal antibody or antigen-binding fragment thereof has: (1a) a heavy chain sequence of SEQ ID NO: 117; and/or, a light chain sequence of SEQ ID NO: 118, or, (2a) a heavy chain sequence of SEQ ID NO: 119; and/or, (2b) a light chain sequence of SEQ ID NO: 120.

Some of the sequences of the antibodies of the invention are provided below.

HFB6-4-hG1 (human-mouse chimeric
monoclonal antibody)
CDR-H1:
(SEQ ID NO: 1)
CTTSGENIKDTYIHW
CDR-H2:
(SEQ ID NO: 2)
GRNDPANGNTKYD
CDR-H3:
(SEQ ID NO: 3)
CASYEGYYEREGYW
CDR-L1:
(SEQ ID NO: 4)
CKSSQSLLDRDGKTYLNW
CDR-L2:
(SEQ ID NO: 5)
LVSKLDS
CDR-L3:
(SEQ ID NO: 6)
CWQDTHEPYTF
HCVR:
(SEQ ID NO: 7)
EVQLQQSGAELVNPGASVKLSCTTSGFNIKDTYIHWVKQRPEQGL
EWIGRNDPANGNTKYDPKFQGKATITADTSSSTAYLQLSSLTSED
TAVYYCASYEGYYERFGYWGQGTLVTVSA
LCVR:
(SEQ ID NO: 8)
DVVMTQAPLTLSVTIGQPASISCKSSQSLLDRDGKTYLNWLLQRP
GQSPKRLIYLVSKLDSGVPDRFTGSGSGTDFTLKISRVEAEDLGV
YYCWQDTHEPYTFGGGTKLEIK
HC:
(SEQ ID NO: 117)
EVQLQQSGAELVNPGASVKLSCTTSGFNIKDTYIHWVKQRPEQGL
EWIGRNDPANGNTKYDPKFQGKATITADTSSSTAYLQLSSLTSED
TAVYYCASYEGYYERFGYWGQGTLVTVSAASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVELFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FELYSKLTVDKSRWQQGNVFSCSVMHEALHNHYTQKSLSLSPGK
LC:
(SEQ ID NO: 118)
DVVMTQAPLTLSVTIGQPASISCKSSQSLLDRDGKTYLNWLLQRP
GQSPKRLIYLVSKLDSGVPDRFTGSGSGTDFTLKISRVEAEDLGV
YYCWQDTHEPYTFGGGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSENRGEC
HFB6-5-hG1 (human-mouse chimeric
monoclonal antibody)
CDR-H1:
(SEQ ID NO: 61)
CTASGVNIKDTYMYW
CDR-H2:
(SEQ ID NO: 62)
GRIDPANGYTRYD
CDR-H3:
(SEQ ID NO: 63)
CAVYDGYYESFDVW
CDR-L1:
(SEQ ID NO: 64)
CKSSQSLLDSDGKTYLNW
CDR-L2:
(SEQ ID NO: 65)
LVSKLDS
CDR-L3:
(SEQ ID NO: 66)
CWQGTLFPRTF
HCVR:
(SEQ ID NO: 67)
EVQLQQSGAELVKPGASVKLSCTASGVNIKDTYMYWVKQRPEQGL
EWIGRIDPANGYTRYDPKFQGKATITADTSSNTAHLQLSSLTSED
AAVYYCAVYDGYYESFDVWGAGTTVTVSS
LCVR:
(SEQ ID NO: 68)
DVVMTQAPLTLSVTIGQPASISCKSSQSLLDSDGKTYLNWLLQRP
GQSPKRLIYLVSKLDSGVPDRFTGSGSGTDFTLKINRVEAEDLGV
YYCWQGTLFPRTFGGGTKLEIK
HC:
(SEQ ID NO: 119)
EVQLQQSGAELVKPGASVKLSCTASGVNIKDTYMYWVKQRPEQGL
EWIGRIDPANGYTRYDPKFQGKATITADTSSNTAHLQLSSLTSED
AAVYYCAVYDGYYESFDVWGAGTTVTVSSASTKGPSVFPLAPSSK
STSGGTAALGCLVKDYFPEPVTVSWNSGALTSGVHTFPAVLQSSG
LYSLSSVVTVPSSSLGTQTYICNVNHKPSNTKVDKKVEPKSCDKT
HTCPPCPAPELLGGPSVELFPPKPKDTLMISRTPEVTCVVVDVSH
EDPEVKFNWYVDGVEVHNAKTKPREEQYNSTYRVVSVLTVLHQDW
LNGKEYKCKVSNKALPAPIEKTISKAKGQPREPQVYTLPPSREEM
TKNQVSLTCLVKGFYPSDIAVEWESNGQPENNYKTTPPVLDSDGS
FELYSKLTVDKSRWQQGNVFSCSVMHEALHNHYTQKSLSLSPGK
LC:
(SEQ ID NO: 120)
DVVMTQAPLTLSVTIGQPASISCKSSQSLLDSDGKTYLNWLLQRP
GQSPKRLIYLVSKLDSGVPDRFTGSGSGTDFTLKINRVEAEDLGV
YYCWQGTLFPRTFGGGTKLEIKRTVAAPSVFIFPPSDEQLKSGTA
SVVCLLNNFYPREAKVQWKVDNALQSGNSQESVTEQDSKDSTYSL
SSTLTLSKADYEKHKVYACEVTHQGLSSPVTKSENRGEC

TABLE 1
Humanized variants of HFB6-4 (see
sequence listing for sequences)

| Antibody | VH | VL |
|---|---|---|
| HFB6-4hz1-hG1 | SEQ ID NO: 9 | SEQ ID NO: 10 |
| HFB6-4hz2-hG1 | SEQ ID NO: 11 | SEQ ID NO: 12 |
| HFB6-4hz3-hG1 | SEQ ID NO: 13 | SEQ ID NO: 14 |
| HFB6-4hz4-hG1 | SEQ ID NO: 15 | SEQ ID NO: 16 |
| HFB6-4hz5-hG1 | SEQ ID NO: 17 | SEQ ID NO: 18 |
| HFB6-4hz6-hG1 | SEQ ID NO: 19 | SEQ ID NO: 20 |
| HFB6-4hz7-hG1 | SEQ ID NO: 21 | SEQ ID NO: 22 |
| HFB6-4hz8-hG1 | SEQ ID NO: 23 | SEQ ID NO: 24 |
| HFB6-4hz9-hG1 | SEQ ID NO: 25 | SEQ ID NO: 26 |
| HFB6-4hz10-hG1 | SEQ ID NO: 27 | SEQ ID NO: 28 |
| HFB6-4hz11-hG1 | SEQ ID NO: 29 | SEQ ID NO: 30 |
| HFB6-4hz12-hG1 | SEQ ID NO: 31 | SEQ ID NO: 32 |
| HFB6-4hz13-hG1 | SEQ ID NO: 33 | SEQ ID NO: 34 |
| HFB6-4hz14-hG1 | SEQ ID NO: 35 | SEQ ID NO: 36 |
| HFB6-4hz15-hG1 | SEQ ID NO: 37 | SEQ ID NO: 38 |
| HFB6-4hz16-hG1 | SEQ ID NO: 39 | SEQ ID NO: 40 |
| HFB6-4hz17-hG1 | SEQ ID NO: 41 | SEQ ID NO: 42 |
| HFB6-4hz18-hG1 | SEQ ID NO: 43 | SEQ ID NO: 44 |
| HFB6-4hz19-hG1 | SEQ ID NO: 45 | SEQ ID NO: 46 |
| HFB6-4hz20-hG1 | SEQ ID NO: 47 | SEQ ID NO: 48 |
| HFB6-4hz21-hG1 | SEQ ID NO: 49 | SEQ ID NO: 50 |
| HFB6-4hz22-hG1 | SEQ ID NO: 51 | SEQ ID NO: 52 |
| HFB6-4hz23-hG1 | SEQ ID NO: 53 | SEQ ID NO: 54 |
| HFB6-4hz24-hG1 | SEQ ID NO: 55 | SEQ ID NO: 56 |

TABLE 2
Humanized variants of HFB6-5 (see
sequence listing for sequences)

| Antibody | VH | VL |
|---|---|---|
| HFB6-5hz1-hG1 | SEQ ID NO: 69 | SEQ ID NO: 70 |
| HFB6-5hz2-hG1 | SEQ ID NO: 71 | SEQ ID NO: 72 |
| HFB6-5hz3-hG1 | SEQ ID NO: 73 | SEQ ID NO: 74 |
| HFB6-5hz4-hG1 | SEQ ID NO: 75 | SEQ ID NO: 76 |
| HFB6-5hz5-hG1 | SEQ ID NO: 77 | SEQ ID NO: 78 |
| HFB6-5hz6-hG1 | SEQ ID NO: 79 | SEQ ID NO: 80 |
| HFB6-5hz7-hG1 | SEQ ID NO: 81 | SEQ ID NO: 82 |
| HFB6-5hz8-hG1 | SEQ ID NO: 83 | SEQ ID NO: 84 |
| HFB6-5hz9-hG1 | SEQ ID NO: 85 | SEQ ID NO: 86 |
| HFB6-5hz10-hG1 | SEQ ID NO: 87 | SEQ ID NO: 88 |
| HFB6-5hz11-hG1 | SEQ ID NO: 89 | SEQ ID NO: 90 |
| HFB6-5hz12-hG1 | SEQ ID NO: 91 | SEQ ID NO: 92 |
| HFB6-5hz13-hG1 | SEQ ID NO: 93 | SEQ ID NO: 94 |
| HFB6-5hz14-hG1 | SEQ ID NO: 95 | SEQ ID NO: 96 |
| HFB6-5hz15-hG1 | SEQ ID NO: 97 | SEQ ID NO: 98 |
| HFB6-5hz16-hG1 | SEQ ID NO: 99 | SEQ ID NO: 100 |
| HFB6-5hz17-hG1 | SEQ ID NO: 101 | SEQ ID NO: 102 |
| HFB6-5hz18-hG1 | SEQ ID NO: 103 | SEQ ID NO: 104 |
| HFB6-5hz19-hG1 | SEQ ID NO: 105 | SEQ ID NO: 106 |
| HFB6-5hz20-hG1 | SEQ ID NO: 107 | SEQ ID NO: 108 |
| HFB6-5hz21-hG1 | SEQ ID NO: 109 | SEQ ID NO: 110 |
| HFB6-5hz22-hG1 | SEQ ID NO: 111 | SEQ ID NO: 112 |
| HFB6-5hz23-hG1 | SEQ ID NO: 113 | SEQ ID NO: 114 |
| HFB6-5hz24-hG1 | SEQ ID NO: 115 | SEQ ID NO: 116 |

In some embodiment, the monoclonal antibodies of the invention or antigen-binding fragments thereof are human-mouse chimeric antibodies, humanized antibodies, human antibodies, CDR-grafted antibodies, or resurfaced antibodies.

In certain embodiments, the isolated monoclonal antibody or antigen-binding fragment thereof is an IgG1 antibody, an IgG2 antibody, an IgG3 antibody, or an IgG4 antibody.

In certain embodiments, the isolated monoclonal antibody or antigen-binding fragment thereof is an IgG1 antibody.

In certain embodiments, the isolated monoclonal antibody or antigen-binding fragment thereof is an IgG1 antibody comprising a heavy chain constant region sequence of SEQ ID NO: 57 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein, and a light chain constant region sequence of SEQ ID NO: 58 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein.

In certain embodiments, the isolated monoclonal antibody or antigen-binding fragment thereof is an IgG1 antibody comprising a mutation in the heavy chain constant region that: (1) modulates antibody dependent cell mediated cytotoxicity (ADCC) and/or antibody dependent cell mediated phagocytosis (ADCP) (such as F243L, G236A, S239D/I332E, S239D/A330L/I332E, S298A/E333A/K334A, F243L/R292P/Y300L/V305I/P396L, or afucosylated (non-fucosylated) antibody (such as afucosylated N297 at Fc region) with enhanced ADCC through increased binding to FcγRIIIa); and/or, (2) enhances serum half-life (such as T250Q/M428L, M252Y/S254T/T256E).

In certain embodiments, the isolated monoclonal antibody or antigen-binding fragment thereof is a mouse antibody, a human-mouse chimeric antibody, a humanized antibody, a human antibody, a CDR-grafted antibody, or a resurfaced antibody.

In certain embodiments, the isolated monoclonal antibody or antigen-binding fragment thereof is a humanized antibody.

In some embodiments, the antigen-binding fragment thereof is an Fab, Fab′, F(ab′)₂, F_{d}, single chain Fv or scFv, disulfide linked Fᵥ, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

In certain embodiments, the monoclonal antibodies of the invention or antigen-binding fragments thereof are specific for human BTLA, e.g., substantially do not cross-react with mouse BTLA.

In certain embodiments, the monoclonal antibodies of the invention or antigen-binding fragments thereof cross-react with a non-human primary BTLA, such as a monkey BTLA (e.g., a cynomolgus monkey or rhesus monkey BTLA, such as SEQ ID NO: 122). In certain embodiments, the monoclonal antibodies of the invention or antigen-binding fragments thereof do not cross-react with a rodent BTLA, such as a mouse BTLA (such as SEQ ID NO: 123). Exemplary amino acid sequences known in the art of human, monkey and mouse BTLA are listed below.

| | BTLA sequence |
|---|---|
| Human BTLA | MKTLPAMLGTGKLFWVFFLIPYLDI |
| (Accession No. | WNIHGKESCDVQLYIKRQSEHSILA |
| NP_861445.3) | GDPFELECPVKYCANRPHVTWCKLN |
| | GTTCVKLEDRQTSWKEEKNISFFIL |
| | HFEPVLPNDNGSYRCSANFQSNLIE |
| | SHSTTLYVTDVKSASERPSKDEMAS |
| | RPWLLYSLLPLGGLPLLITTCFCLF |
| | CCLRRHQGKQNELSDTAGREINLVD |
| | AHLKSEQTEASTRQNSQVLLSETGI |
| | YDNDPDLCFRMQEGSEVYSNPCLEE |
| | NKPGIVYASLNHSVIGPNSRLARNV |
| | KEAPTEYASICVRS |
| | (SEQ ID NO: 121) |
| Cynomolgus/ | MKTLPAMLGSGRLFWVVFLIPYLDI |
| Rhesus Macaque | WNIHGKESCDVQLYIKRQSYHSIFA |
| BTLA | GDPFKLECPVKYCAHRPQVTWCKLN |
| (Accession No. | GTTCVKLEGRHTSWKQEKNLSFFIL |
| XP_001104281.1) | HFEPVLPSDNGSYRCSANFLSAIIE |
| | SHSTTLYVTDVKSASERPSKDEMAS |
| | RPWLLYSLLPLGGLPLLITTCFCLF |
| | CFLRRHQGKQNELSDTTGREITLVD |
| | VPFKSEQTEASTRQNSQVLLSETGI |
| | YDNEPDFCFRMQEGSEVYSNPCLEE |
| | NKPGIIYASLNHSIIALNSRQARNV |
| | KEAPTEYASICVRS |
| | (SEQ ID NO: 122) |
| Mouse BTLA | MKTVPAMLGTPRLFREFFILHLGLW |
| (Accession No. | SILCEKATKRNDEECPVQLTITRNS |
| NP_001032808.2) | KQSARTGELFKIQCPVKYCVHRPNV |
| | TWCKHNGTICVPLEVSPQLYTSWEE |
| | NQSVPVFVLHFKPIHLSDNGSYSCS |
| | TNFNSQVINSHSVTIHVTERTQNSS |
| | EHPLITVSDIPDATNASGPSTMEER |
| | PGRTWLLYTLLPLGALLLLLACVCL |
| | LCFLKRIQGKEKKPSDLAGRDTNLV |
| | DIPASSRTNHQALPSGTGIYDNDPW |
| | SSMQDESELTISLQSERNNQGIVYA |
| | SLNHCVIGRNPRQENNMQEAPTEYA |
| | SICVRS (SEQ ID NO: 123) |

In some embodiments, the monoclonal antibody of the invention or antigen-binding fragment thereof has a dissociation constant (K_{D}) of ≤1 μM, ≤100 nM, ≤50 nM, ≤25 nM, ≤20 nM, ≤15 nM, ≤10 nM, ≤5 nM, ≤2 nM, ≤1 nM, ≤0.1 nM, ≤0.01 nM, or ≤0.001 nM (e.g. 10⁻⁸ M or less, e.g. from 10⁻⁸ M to 10⁻¹³ M, e.g., from 10⁻⁹ M to 10⁻¹³ M) for human BTLA.

In some embodiments, the monoclonal antibody of the invention or antigen-binding fragment thereof binds to the epitope bound by HFB6-4. In certain embodiment, the monoclonal antibody of the invention or antigen-binding fragment thereof binds to the epitope bound by HFB6-5.

In some embodiments, the monoclonal antibody of the invention or antigen-binding fragment thereof is an agonist of human BTLA and activates downstream signaling from BTLA upon binding to BTLA.

In some embodiments, said downstream signaling from BTLA inhibits B cell proliferation, and/or inhibits T cell (e.g., CD4, CD8, Th1, TFH, αβ, or γδ T-cell) and/or plasma cell activation

In some embodiments, the monoclonal antibody of the invention or antigen-binding fragment thereof does not block/interfere/abolish BTLA binding to HVEM.

In some embodiments, the monoclonal antibody of the invention or antigen-binding fragment thereof inhibits B cell proliferation, and/or inhibits T cell (e.g., CD4, CD8, Th1, TFH, αβ, or γδ T-cell) activation, upon binding to BTLA.

In certain embodiments, the isolated monoclonal antibody or antigen-binding fragment thereof of the invention, comprises: (1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or, (2) the heavy chain amino acid sequence of SEQ ID NO: 59, and the light chain amino acid sequence of SEQ ID NO: 60.

In a related aspect, the invention provides an isolated monoclonal antibody or antigen-binding fragment thereof, comprising/consisting essentially of/consisting of: (1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or, (2) the heavy chain amino acid sequence of SEQ ID NO: 59, and the light chain amino acid sequence of SEQ ID NO: 60.

In some embodiments, the monoclonal antibodies of the invention or antigen-binding fragments thereof, including humanized monoclonal antibodies or antigen-binding fragments thereof, have good developability profile, including being stable under high temperature (e.g., stable at storage at 25° C. or 40° C.), low pH conditions (e.g., pH3.5 around room temperature), and/or following several rounds of freeze/thaw cycles.

In certain embodiments, the monoclonal antibodies of the invention or antigen-binding fragments thereof, including humanized monoclonal antibodies or antigen-binding fragments thereof, include one or more point mutations of in amino acid sequences that are designed to improve developability of the antibody. For example, Raybould et al. (Five computational developability guidelines for therapeutic antibody profiling, PNAS 116(10): 4025-4030, 2019) described Therapeutic Antibody Profiler (TAP), a computational tool that builds downloadable homology models of variable domain sequences, tests them against five developability guidelines, and reports potential sequence liabilities and canonical forms. The authors further provide TAP as freely available at opig.stats.ox.ac.uk/webapps/sabdab-sabpred/TAP.php.

There are many barriers to therapeutic mAb development, besides achieving the desired affinity to the antigen. These include intrinsic immunogenicity, chemical and conformational instability, self-association, high viscosity, polyspecificity, and poor expression. For example, high levels of hydrophobicity, particularly in the highly variable complementarity-determining regions (CDRs), have repeatedly been implicated in aggregation, viscosity, and polyspecificity. Asymmetry in the net charge of the heavy- and light-chain variable domains is also correlated with self-association and viscosity at high concentrations. Patches of positive and negative charge in the CDRs are linked to high rates of clearance and poor expression levels. Product heterogeneity (e.g., through oxidation, isomerization, or glycosylation) often results from specific sequence motifs liable to post- or co-translational modification. Computational tools are available to facilitate the identification of sequence liabilities. Warszawski et al. (Optimizing antibody affinity and stability by the automated design of the variable light-heavy chain interfaces. PLoS Comput Biol 15(8): e1007207. https://doi.org/10.1371/journal.pcbi.1007207) also described methods of optimizing antibody affinity and stability by an automated design of the variable light-heave chain interfaces. Additional methods are available to identify potential developability issues of a candidate antibody, and in preferred embodiments of this invention, one or more point mutations can be introduced, via conventional methods, to the candidate antibody to address such issues to lead to an optimized therapeutic antibody of the invention.

### 7. Humanized Antibodies

In some embodiments, the antibody of the invention is a humanized antibody. Humanized antibodies are useful as therapeutic molecules because humanized antibodies reduce or eliminate the human immune response to non-human antibodies (such as the human anti-mouse antibody (HAMA) response), which can result in an immune response to an antibody therapeutic, and decreased effectiveness of the therapeutic.

An antibody may be humanized by any standard method. Non-limiting exemplary methods of humanization include methods described, e.g., in U.S. Pat. Nos. 5,530,101; 5,585,089; 5,693,761; 5,693,762; 6,180,370; Jones et al., Nature 321:522-525 (1986); Riechmann et al, Nature 332: 323-27 (1988); Verhoeyen et al, Science 239: 1534-36 (1988); and U.S. Publication No. US 2009/0136500. All incorporated by reference.

A humanized antibody is an antibody in which at least one amino acid in a framework region of a non-human variable region has been replaced with the amino acid from the corresponding location in a human framework region. In some embodiments, at least two, at least three, at least four, at least five, at least six, at least seven, at least eight, at least nine, at least 10, at least 11, at least 12, at least 15, or at least 20 amino acids in the framework regions of a non-human variable region are replaced with an amino acid from one or more corresponding locations in one or more human framework regions.

In some embodiments, some of the corresponding human amino acids used for substitution are from the framework regions of different human immunoglobulin genes. That is, in some such embodiments, one or more of the non-human amino acids may be replaced with corresponding amino acids from a human framework region of a first human antibody or encoded by a first human immunoglobulin gene, one or more of the non-human amino acids may be replaced with corresponding amino acids from a human framework region of a second human antibody or encoded by a second human immunoglobulin gene, one or more of the non-human amino acids may be replaced with corresponding amino acids from a human framework region of a third human antibody or encoded by a third human immunoglobulin gene, etc. Further, in some embodiments, all of the corresponding human amino acids being used for substitution in a single framework region, for example, FR2, need not be from the same human framework. In some embodiments, however, all of the corresponding human amino acids being used for substitution are from the same human antibody or encoded by the same human immunoglobulin gene.

In some embodiments, an antibody is humanized by replacing one or more entire framework regions with corresponding human framework regions. In some embodiments, a human framework region is selected that has the highest level of homology to the non-human framework region being replaced. In some embodiments, such a humanized antibody is a CDR-grafted antibody.

In some embodiments, following CDR-grafting, one or more framework amino acids are changed back to the corresponding amino acid in a mouse framework region. Such “back mutations” are made, in some embodiments, to retain one or more mouse framework amino acids that appear to contribute to the structure of one or more of the CDRs and/or that may be involved in antigen contacts and/or appear to be involved in the overall structural integrity of the antibody. In some embodiments, ten or fewer, nine or fewer, eight or fewer, seven or fewer, six or fewer, five or fewer, four or fewer, three or fewer, two or fewer, one, or zero back mutations are made to the framework regions of an antibody following CDR grafting.

In some embodiments, a humanized antibody also comprises a human heavy chain constant region and/or a human light chain constant region.

In some embodiments, the humanized anti-BTLA antibody or antigen-binding fragment thereof of the invention is one the antibodies listed in Tables 1-2 and the accompanying sequence listing (incorporated herein as part of the specification).

### 8. Human Antibodies

In some embodiments, the antibody of the invention is a human antibody. Human antibodies can be made by any suitable method. Non-limiting exemplary methods include making human antibodies in transgenic mice that comprise human immunoglobulin loci.

See, e.g., Jakobovits et al., Proc. Natl. Acad. Sci. USA 90: 2551-55 (1993); Jakobovits et al, Nature 362: 255-8 (1993); onberg et al, Nature 368: 856-9 (1994); and U.S. Pat. Nos. 5,545,807; 6,713,610; 6,673,986; 6,162,963; 5,545,807; 6,300,129; 6,255,458; 5,877,397; 5,874,299; and 5,545,806.

Non-limiting exemplary methods also include making human antibodies using phage display libraries. See, e.g., Hoogenboom et al., J. Mol. Biol. 227: 381-8 (1992); Marks et al, J. Mol. Biol. 222: 581-97 (1991); and PCT Publication No. WO 99/10494.

### Antibody Constant Regions

In some embodiments, a humanized, chimeric, or human antibody described herein comprises one or more human constant regions. In some embodiments, the human heavy chain constant region is of an isotype selected from IgA, IgG, and IgD. In some embodiments, the human light chain constant region is of an isotype selected from K and λ. In some embodiments, an antibody described herein comprises a human IgG constant region, for example, human IgG1, IgG2, IgG3, or IgG4. In some embodiments, an antibody or Fc fusion partner comprises a C237S mutation, for example, in an IgG1 constant region. In some embodiments, an antibody described herein comprises a human IgG2 heavy chain constant region. In some such embodiments, the IgG2 constant region comprises a P331S mutation, as described in U.S. Pat. No. 6,900,292. In some embodiments, an antibody described herein comprises a human IgG4 heavy chain constant region. In some such embodiments, an antibody described herein comprises an S241P mutation in the human IgG4 constant region. See, e.g., Angal et al. Mol. Immunol. 30(1):105-108 (1993). In some embodiments, an antibody described herein comprises a human IgG4 constant region and a human κ light chain.

In some embodiments, the isolated monoclonal antibody of the invention or antigen-binding fragment thereof comprises a human IgG1 constant region, comprising a heavy chain constant region sequence of SEQ ID NO: 57 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein, and a light chain constant region sequence of SEQ ID NO: 58 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein.

The choice of heavy chain constant region can determine whether or not an antibody will have effector function in vivo. Such effector function, in some embodiments, includes antibody-dependent cell-mediated cytotoxicity (ADCC) and/or complement-dependent cytotoxicity (CDC), and can result in killing of the cell to which the antibody is bound. Typically, antibodies comprising human IgG1 or IgG3 heavy chains have effector function.

In some embodiments, the isolated monoclonal antibody of the invention or antigen-binding fragment thereof is an IgG1 antibody comprising a mutation in the heavy chain constant revion that modulates antibody dependent cell mediated cytotoxicity (ADCC) and/or antibody dependent cell mediated phagocytosis (ADCP) (such as F243L, G236A, S239D/1332E, S239D/A330L/1332E, F243L/R292P/Y300L/V305I/P396L, S298A/E333A/K334A, or afucosylated (non-fucosylated) antibody (such as afucosylated N297 at Fc region) with enhanced ADCC through increased binding to FcγRIIIa).

In some embodiments, the isolated monoclonal antibody of the invention or antigen-binding fragment thereof is an IgG1 antibody comprising a mutation in the heavy chain constant revion that enhances serum half-life (such as T250Q/M428L, and M252Y/S254T/T256E).

In some embodiments, effector function is not desirable. For example, in some embodiments, effector function may not be desirable in treatments of inflammatory conditions and/or autoimmune disorders. In some such embodiments, a human IgG4 or IgG2 heavy chain constant region is selected or engineered. In some embodiments, an IgG4 constant region comprises an S241P mutation.

Any of the antibodies described herein may be purified by any suitable method. Such methods include, but are not limited to, the use of affinity matrices or hydrophobic interaction chromatography. Suitable affinity ligands include the antigen and/or epitope to which the antibody binds, and ligands that bind antibody constant regions. For example, a Protein A, Protein G, Protein A/G, or an antibody affinity column may be used to bind the constant region and to purify an antibody.

In some embodiments, hydrophobic interactive chromatography (HIC), for example, a butyl or phenyl column, is also used for purifying some polypeptides. Many methods of purifying polypeptides are known in the art.

Alternatively, in some embodiments, an antibody described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al, Biotechnol. Adv. 21: 695-713 (2003).

### 9. Nucleic Acid Molecules Encoding Antibodies of the Invention

The invention also provides nucleic acid molecules comprising polynucleotides that encode one or more chains of an antibody described herein. In some embodiments, a nucleic acid molecule comprises a polynucleotide that encodes a heavy chain or a light chain of an antibody described herein. In some embodiments, a nucleic acid molecule comprises both a polynucleotide that encodes a heavy chain and a polynucleotide that encodes a light chain, of an antibody described herein. In some embodiments, a first nucleic acid molecule comprises a first polynucleotide that encodes a heavy chain and a second nucleic acid molecule comprises a second polynucleotide that encodes a light chain.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain and a light chain linked together.

In some embodiments, a polynucleotide encoding a heavy chain or light chain of an antibody described herein comprises a nucleotide sequence that encodes a leader sequence, which, when translated, is located at the N-terminus of the heavy chain or light chain. As discussed above, the leader sequence may be the native heavy or light chain leader sequence, or may be another heterologous leader sequence.

Nucleic acid molecules may be constructed using recombinant DNA techniques conventional in the art. In some embodiments, a nucleic acid molecule is an expression vector that is suitable for expression in a selected host cell, such as a mammalian cell.

In some embodiments, a polynucleotide encoding a heavy chain or light chain of an antibody described herein is codon optimized for expression in a human cell.

### 10. Vectors

Vectors comprising polynucleotides that encode heavy chains and/or light chains of the antibodies described herein are provided. Such vectors include, but are not limited to, DNA vectors, phage vectors, viral vectors, retroviral vectors, etc. In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain and light chain are expressed as part of a single polypeptide, such as, for example, when the antibody is an scFv.

In some embodiments, a vector comprising polynucleotides that encode heavy chains and/or light chains of the antibodies described herein is an expression vector (e.g., a mammalian, yeast, insect, or bacterial expression vector) In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

In some embodiments, a vector is selected that is optimized for expression of polypeptides in CHO or CHO-derived cells, or in NSO cells. Exemplary such vectors are described, e.g., in Running Deer et al., Biotechnol. Prog. 20:880-889 (2004). In some embodiments, a vector is chosen for in vivo expression of the subject antibodies in animals, including humans. In some such embodiments, expression of the polypeptide or polypeptides is under the control of a promoter or promoters that function in a tissue-specific manner. For example, liver-specific promoters are described, e.g., in PCT Publication No. WO 2006/076288.

### 11. Host Cells

In various embodiments, heavy chains and/or light chains of the antibodies described herein may be expressed in prokaryotic cells, such as bacterial cells; or in eukaryotic cells, such as fungal cells (such as yeast), plant cells, insect cells, and mammalian cells. Such expression may be carried out, for example, according to procedures known in the art. Exemplary eukaryotic cells that may be used to express polypeptides include, but are not limited to, COS cells, including COS 7 cells; 293 cells, including 293-6E cells; CHO cells, including CHO—S and DG44 cells; PER.C6® cells (Crucell); and NSO cells. In some embodiments, heavy chains and/or light chains of the antibodies described herein may be expressed in yeast. See, e.g., U.S. Publication No. US 2006/0270045 A1. In some embodiments, a particular eukaryotic host cell is selected based on its ability to make desired post-translational modifications to the heavy chains and/or light chains of the anti-BTLA antibodies. For example, in some embodiments, CHO cells produce polypeptides that have a higher level of sialylation than the same polypeptide produced in 293 cells.

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc., Nonlimiting exemplary methods are described, e.g., in Sambrook et al., Molecular Cloning, A Laboratory Manual, 3rd ed. Cold Spring Harbor Laboratory Press (2001). Nucleic acids may be transiently or stably transfected in the desired host cells, according to any suitable method.

In some embodiments, one or more polypeptides may be produced in vivo in an animal that has been engineered or transfected with one or more nucleic acid molecules encoding the polypeptides, according to any suitable method.

### EXAMPLES

### Example 1 Identification of Human BTLA Agonist Antibodies that Cross-Reacts with Cynomolgus Monkey BTLA

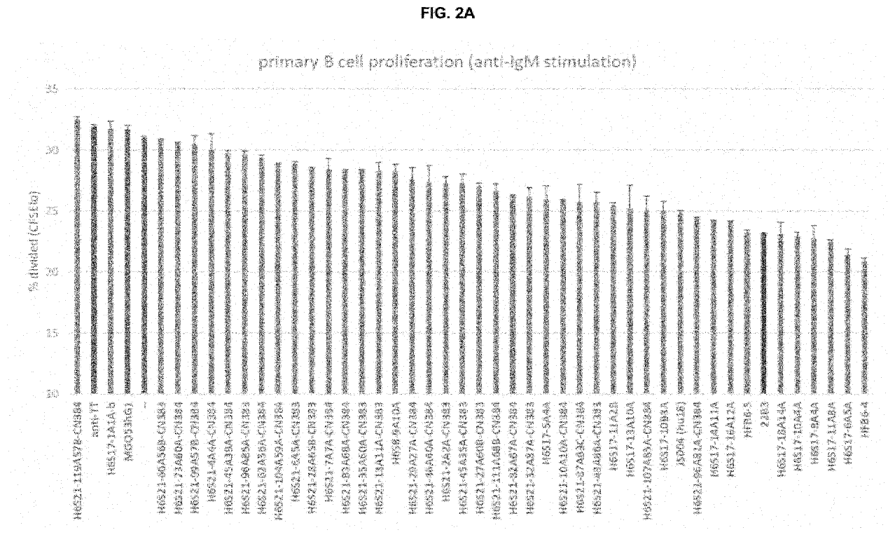

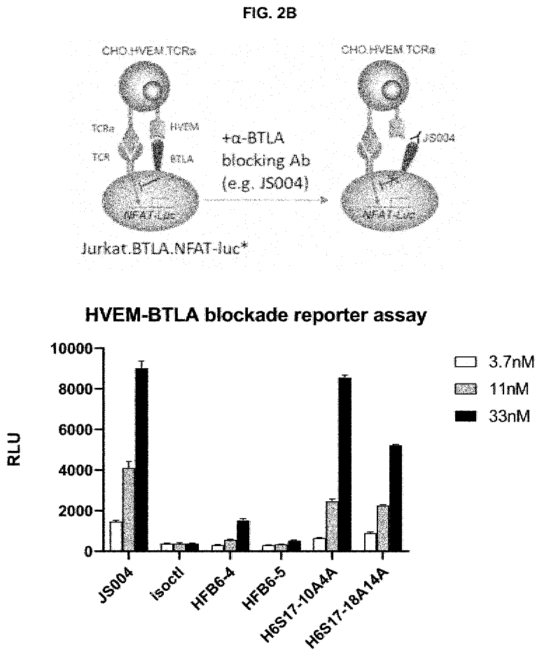

This example describes the identification of two mouse lead antibody candidates—HFB6-4 and HFB6-5—based on their ability to inhibit anti-IgM-mediated B cell proliferation to a similar or greater extent than a benchmark BTLA agonist antibody (referred to herein as 22B3), (FIG. 2A) as well as their inability to reverse HVEM-mediated T cell inhibition (FIG. 2B). Thus, these mouse antibodies are sometimes referred to as “agonist non-blocker,” due to their ability to bind to BTLA and initiate the immune-suppressive function downstream of BTLA, as well as their inability to displace the natural ligand of BTLA (i.e., HVEM) when bound to BTLA.

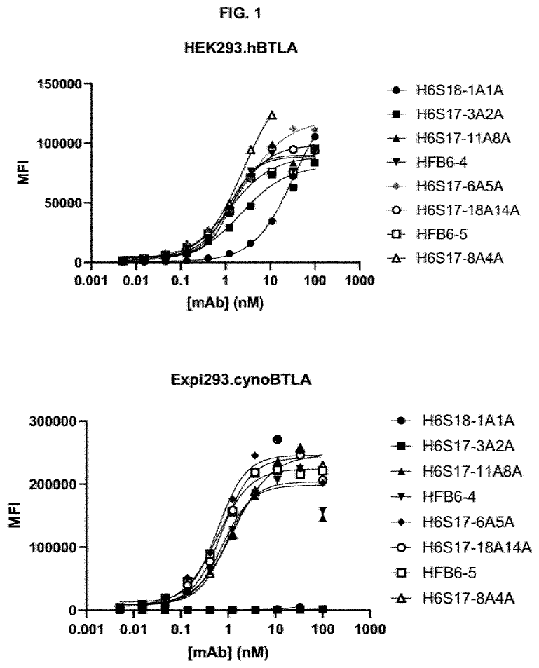

In FIG. 1, HEK293 cells overexpressing human BTLA and Expi293 cells overexpressing cynomolus BTLA were incubated with different concentration of anti-BTLA parental antibodies. Anti-BTLA antibody binding to target cells were quantified with an AF647-conjugated anti-human IgG Fc secondary antibody by flow cytometry. The HFB6-4 and HFB6-5 parental antibodies were found to bind to human BTLA and cyno BTLA with single digit nM affinities.

In FIG. 2A, primary B cells were isolated from healthy donor PBMC using EasySep™ Human B Cell Isolation Kit and labeled with CFSE. B cells were stimulated with 10 μg/mL anti-IgM in the presence of 100 nM anti-BTLA antibodies or control antibodies. After 4 days, B cell proliferation was quantified by CFSE dilution (% CFSElo) related to nonstimulated B cells.

In the HVEM-BTLA blockade reporter assay, a CHO cell line enginnered to express a TCR activator (TCRa) and the BTLA ligand HVEM was co-cultured with a Jurkat reporter cell line that expresses BTLA and a luciferase reporter driven by NFAT binding promoter. Application of a benchmark anti-BTLA blocking antibody (referred to herein as JS004) could reverse HVEM-mediated T cell inhibition, but not HFB6-4 and HFB6-5 (FIG. 2B).

It was further confirmed that the HFB6-4 and 22B3 epitopes overlap at BTLA/HVEM binding interface based on binning experiments. Thus, it is particularly surprising that different antibodies, though binding to apparently the same or substantially overlapping epitopes, appear to have substantially different biological activity.

Specifically, in ELISA binning experiments, ELISA plate was coated with 5 μg/mL of respective capture antibodies (HFB6-4 or 22B3) or ligand (recombinant Fc-tagged HVEM). After blocking, recombinant his-tagged BTLA (BTLA-his) was administered together with excess amount (30 μg/mL) of competitor antibodies or isotype control antibody (MGO53) in solution. Captured BTLA-his was detected with HRP conjugated anti-his secondary antibody. Percentage of BTLA-his capture inhibition was quantified in relative to using MGO53 as competitor antibody (see table below).

| | | Capture antibody/ligand (5 μg/ml) | | |
|---|---|---|---|---|
| | | HFB6-4 | 22B3 | HVEM-Fc |
| Solution | HFB6-4 | 102 | 67 | 84 |
| antibody | 22B3 | 104 | 87 | 93 |
| (30 μg/ml) | MGO53(c−) | 0 | 0 | 0 |

The data showed that HFB6-4 prevents binding of recombinant BTLA (BTLA-his) to immobilized 22B3, and vice versa, and to recombinant HVEM (HVEM-Fc). These data indicate that HFB6-4 and 22B3 epitopes overlap at the BTLA/HVEM binding interface.

### Example 2 hBTLA Agonist Antibodies HFB6-4 and HFB6-5 Inhibit T Cell Activation

This experiment demonstrates that the isolated HFB6-4 and HFB-6-5 antibodies are functional hBTLA agonists, as evidenced by their ability to inhibit T cell activation.

In this assay, CHO cells expressing FcγRIIb on the cell surface was first incubated with a test antibody, such as HFB6-4 or HFB6-5 (or a positive control or a negative control antibody), with or without an anti-CD3 antibody, such that the test antibody (and the anti-CD3 antibody if added) were bound to the surface FcγRIIb via the Fc region.

The test antibody-covered CHO cells were then incubated with a Jurkat reporter cell expressing surface TCR and BTLA. When the anti-CD3 antibody was also present on the CHO cells, upon TCR activation by the anti-CD3 antibody, a BTLA agonist antibody such as HFB6-4 and HFB6-5 engaged BTLA and stimulated downstream BTLA signaling, thus inhibiting TCR signaling, as measured in the reporter cell by a luciferase reporter gene under the transcriptional control of a TCR-downstream NFAT promoter.

No luciferase activity was detected in a negative control when no anti-CD3 antibody was added to stimulate TCR activation. Luciferase activity was not attenuated in the presence of a control antibody MGO that did not bind BTLA.

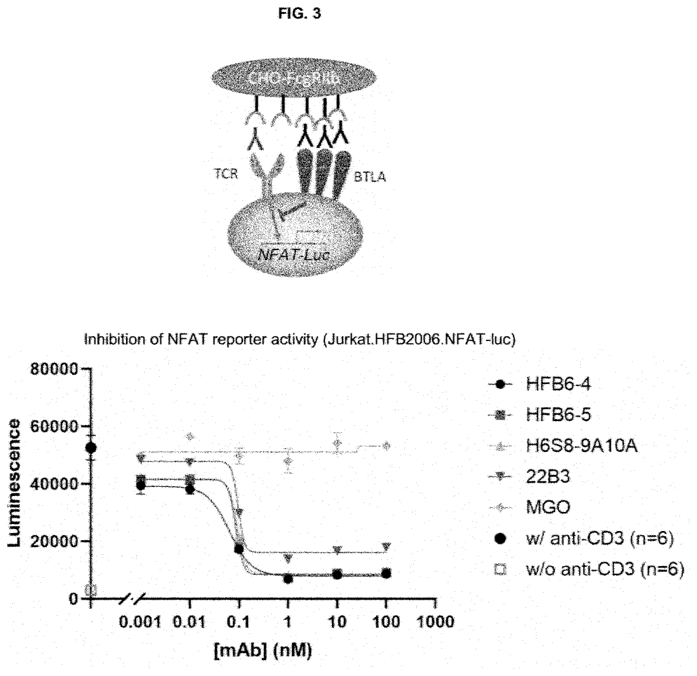

In contrast, a benchmark BTLA agonist antibody 22B3 reduced/inhibited TCR-mediated luciferase activity in a dose dependent manner. Both HFB6-4 and HFB6-5, as well as another BTLA agonist antibody H6S8-9A10A, inhibited TCR-mediated luciferase activity to a larger extent than the benchmark antibody 22B3. See FIG. 3.

The data demonstrated that HFB6-4, HFB6-5, and H6S8-9A10A were at least as good as (if not better than) the benchmark agonist antibody 22B3 in inhibiting TCR signaling.

### Example 3 hBTLA Agonist Antibodies HFB6-4/HFB6-5 Inhibits B Cell Proliferation

This example demonstrates that the BTLA agonist antibodies HFB6-4 and HFB6-5 inhibit primary B cell proliferation to an extent comparable to the benchmark agonist antibody 22B3 in all donors tested.

Primary B cells were isolated from healthy donor PBMC using EasySep™ Human B Cell Isolation Kit and labeled with CFSE. B cells were stimulated with 10 μg/mL anti-IgM in the presence of anti-BTLA antibodies or control antibodies. After 4 days, B cell proliferation was quantified by CFSE dilution (% CFSElo) related to nonstimulated B cells.

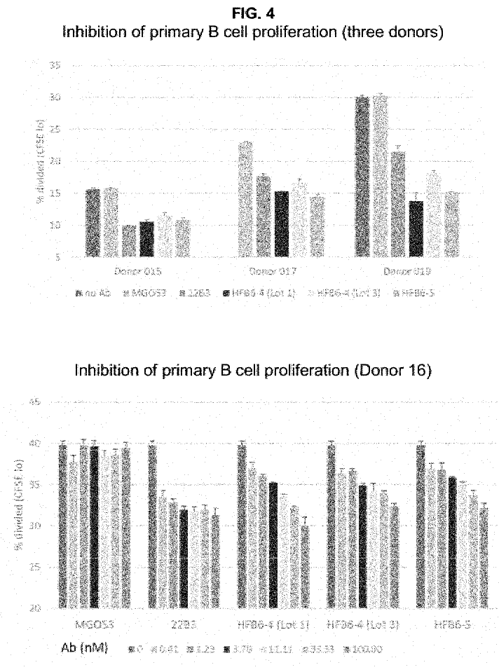

HFB6-4 and HFB6-5 inhibited proliferation of primary B cell from 3 different donors to a similar level (donor 015 and donor 017) or better (donor 019) than the benchmark 22B3 antibody (FIG. 4, left panel). Increasing concentrations of the HFB6-4 and HFB6-5 also led to increased inhibition of primary B cell proliferation (FIG. 4, right panel).

### Example 4 Binding and Cross-reactivity of Humanized HFB6-4/HFB6-5 Variants

This experiment demonstrates that the humanized variants of HFB6-4 and HFB6-5 all bind to hBTLA and cross-reacts with Cynomolgus monkey BTLA with high affinity, as the parental HFB6-4 and HFB6-5 antibodies.

Briefly, 24 each humanized variants of HFB6-4 and HFB6-5 (e.g., HFB6-4-hz1 to -hz24, and HFB6-5-hz1 to -hz24) were generated based on standard humanization protocols, and the resulting humanized variants were tested to identify high affinity binders to hBTLA and cynoBTLA.

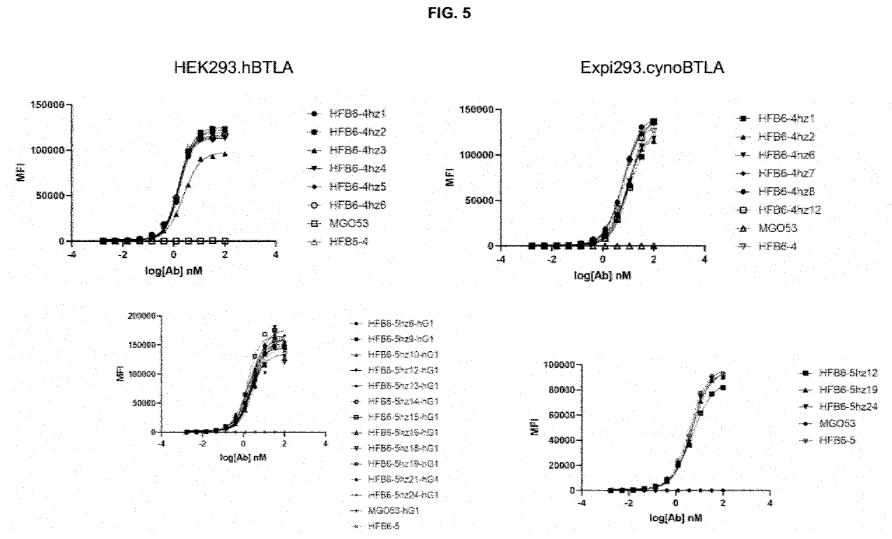

HEK293 cells overexpressing human BTLA and Expi293 cells overexpressing cynomolus BTLA were incubated with different concentrations of humanized anti-BTLA antibodies. Anti-BTLA antibody binding to target cells were quantified with an AF647-conjugated anti-human IgG Fc secondary antibody by flow cytometry. See FIG. 5

The results show that the HFB6-4 and HFB6-5 humanized variants bind human and cynomolgus BTLA with single digit nM affinities.

### Example 5 HFB6-4 and HFB6-5 Humanized Variants Inhibit B Cell Proliferation and T-cell Reporter Activity

The capability of selected HFB6-4 and HFB6-5 humanized variants in inhibiting B cell proliferation and T-cell reporter activity was compared with that of parental antibodies and benmark controls.

In the B cell proliferation suppression assay, primary B cells were isolated from healthy donor PBMC using EasySep™ Human B Cell Isolation Kit and labeled with CFSE. B cells were stimulated with 10 μg/mL anti-IgM in the presence of 1 μg/mL and 10 μg/mL anti-BTLA antibodies or control antibodies. After 4 days, B cell proliferation was quantified by CFSE dilution (% CFSElo) related to nonstimulated B cells.

In the T-cell suppression reporter assay, a CHO cell line engineered to express FcγRIIb were used to cross-link anti-CD3 and anti-BTLA antibodies. Upon Fc receptor crossing, anti-BTLA agonist antibodies could inhibit luciferase reporter signal driven by TCR-NFAT pathway activation in the Jurkat reporter cell line expressing BTLA and NFAT luciferase reporter.

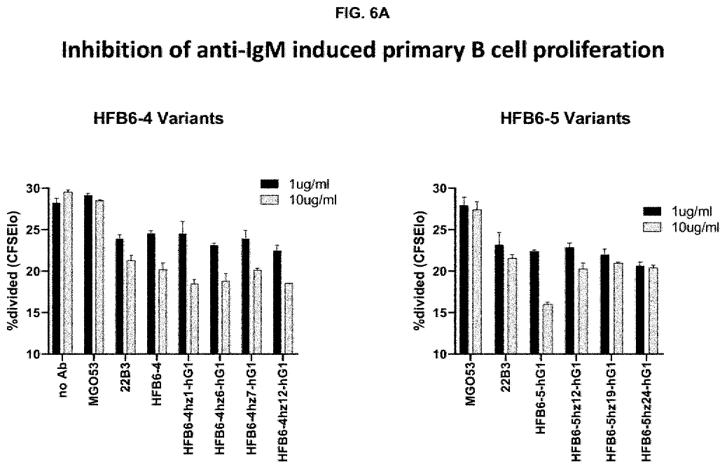

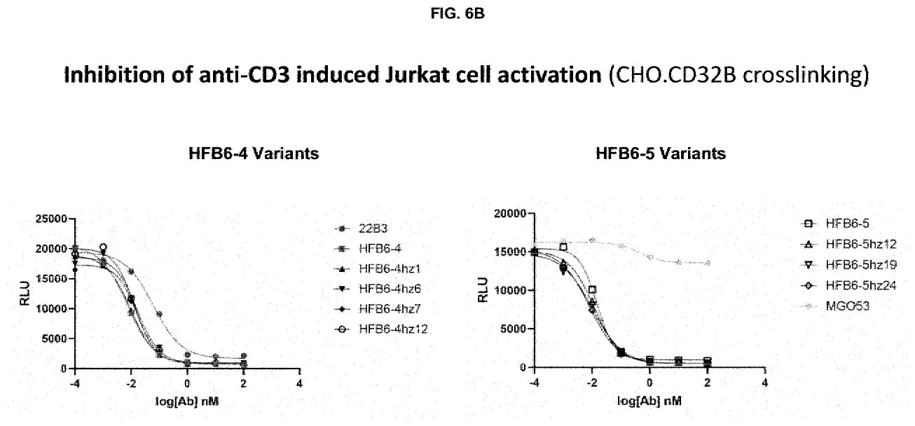

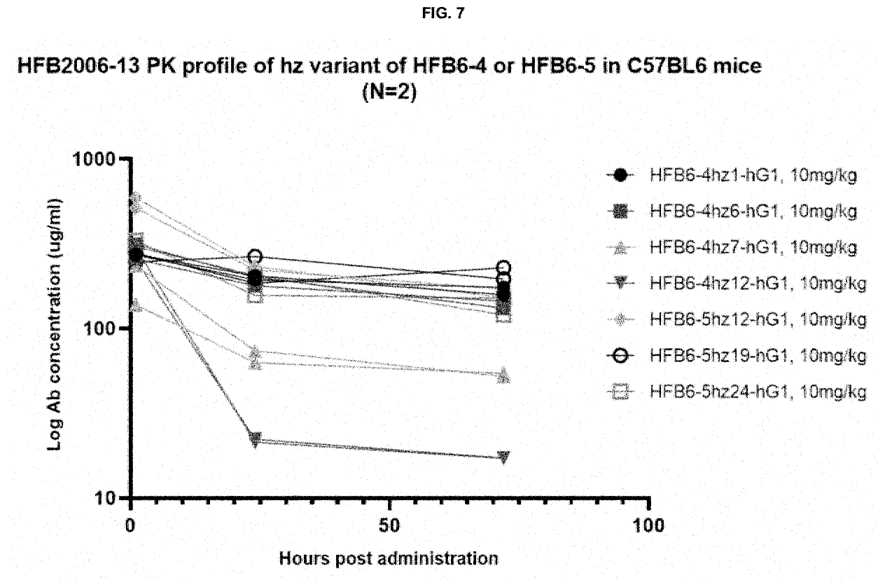

The result shows that selected HFB6-4 and HFB6-5 humanized variants maintain parental HFB6-4 and HFB6-5 activity in inhibiting B cell proliferation (FIG. 6A) and T cell reporter activity (FIG. 6B).

### Example 6 Desirable PK Profiles of Selected HFB6-4 and HFB6-5 Humanized Variants in Wild-type C57BL/6 Mice

To examine pharmacokinetic profiles of the HFB6-4 and HFB6-5 humanized variants, single dose (10 mg/kg) of selected variants was intravenously injected into wild type C57BL/6 mice. At 1 hr, 24 hrs, and 72 hrs, blood was drawn and plasma concentration of selected antibodies were measured by ELISA.

The data shows that selected humanized variants, such as HFB6-4 humanized variants HFB6-4hz1-hG1, HFB6-4hz6-hG1, and HFB6-5 humanized variants HFB6-5hz12-hG1, HFB6-5hz19-hG1 and HFB6-5hz24-hG1, exhibited prolonged exposures in mice, when administered as 10 mg/kg single dose.

### Example 7 Favorable Developability of Selected Humanized Variants

Developability of selected humanized variants of HFB6-4 and HFB6-5 were examined, and the variants exhibit favorable developability characteristics in multiple assays, such as the melting temperature assay, stability at 4° C. and 37° C., and stability at low pH (e.g., pH3.8).

### Example 8 Therapeutic Efficacy in hPBMC-NSG Acute GvHD Model

This example demonstrates that the subject BTLA agonist antibodies are effective to inhibit immune reaction in an NSG mouse model of acute GvHD (graft v. host disease).

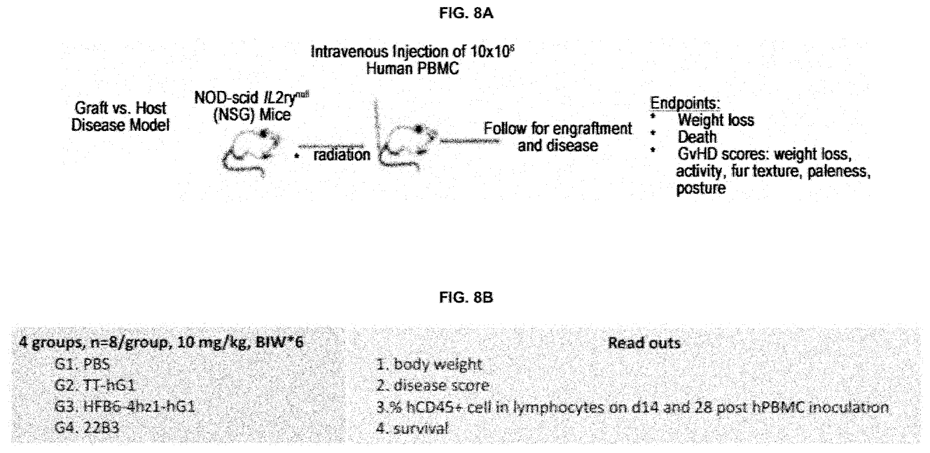

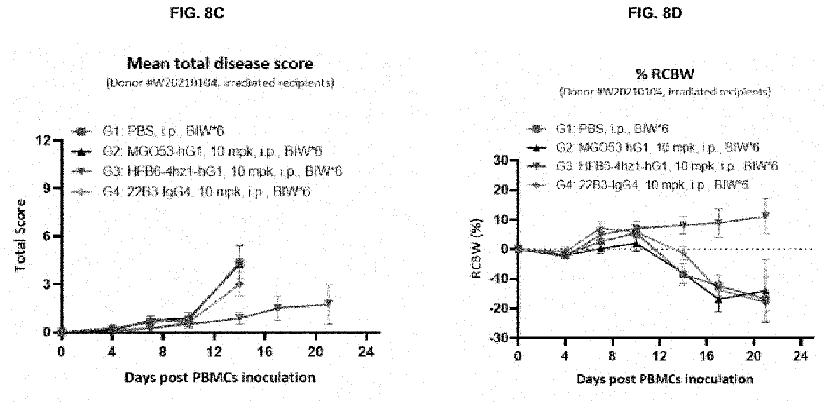

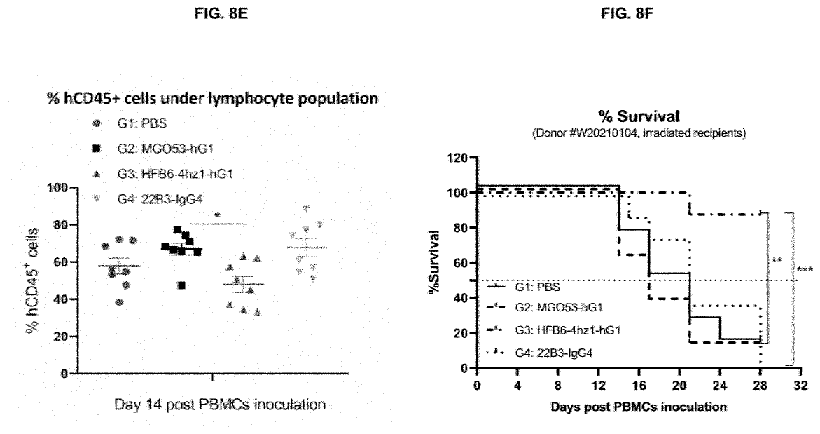

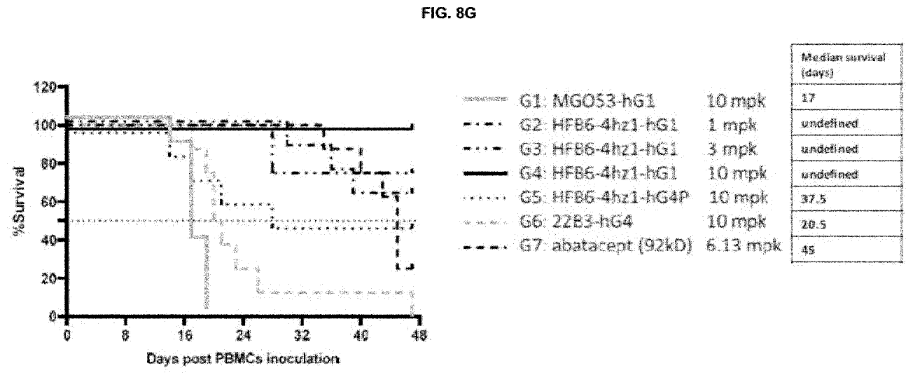

In this experiment, in vivo efficacy of the subject BTLA agonist antibodies is evaluated in a xenogeneic humanized mouse model of acute graft versus host disease (GvHD). This mouse model is a T cell-mediated disease induced by engagement of human PBMCs in sub-lethally irradiated NSG mice. A schematic drawing of the experimental protocol is illustrated in FIG. 8A.

The FIG. 8B shows the more detailed experimental design, including doses for each treatment group, and the read-outs to be measured. Each of the 4 treatment groups has 8 mice. They include the PBS negative control group (G1), the IgG1 isotype-matched control group (MGO53-hG1, G2), the BTLA agonist antibody (HFB6-4-hz1-hG1) group (G3), and the 22B3 benchmark control group (G4).

Preliminary data showed that, at Day 14 post hPBMC inoculation, G3 mice treated with the subject BTLA agonist antibody HFB6-4-hz1-hG1 exhibited statistically significant reduction of hCD45+ immune cell percentage, compared to the isotype-matched control group G2. Meanwhile, the benchmark group G4 did not appear to have a statistically significant difference, if any difference at all, at the same time point. See FIG. 8E.

Furthermore, the Kaplan-Meier survival curve in FIG. 8F shows that more than 85% of the G3 mice treated with the subject BTLA agonist antibody HFB6-4-hz1-hG1 survived at the end of the experiment on Day 28. This is statistically significantly higher than the <20% survival rates of the PBS control group (G1) and the isotype-matched MGO53-hG1 control group (G2).

Meanwhile, survival rate steadily declined after Day 15 in the G4 control treatment group treated by the 22B3 benchmark antibody, such that less than 40% of treated mice survived. The difference in survival rates between the G3 and G4 groups is also statistically significant.

In addition, the HFB6-4hz1-hG1 treatment group (G3) showed significantly reduced total disease score (see FIG. 8C) and relative control body weight (RCBD) loss (FIG. 8D), compared to the PBS control group G1, the isotype-matched control group G2, and the 22B3 benchmark group G4.

The dose response efficacy of HFB6-4hz1-hG1 was also evaluated in the same hPBMC-NSG acute GvHD model at smaller doses of HFB6-4hz1-hG1 compared to the higher dose of the control antibody 22B3 (FIG. 8G). Specifically, treatment with HFB6-4hz1-hG1 at 1, 3, 10 mg/kg all resulted in greater survival benefit than 22B3-hG4 at 10 mg/kg and the IgG4 version of HFB6-4hz1 (HFB6-4hz1-hG4P) at 10 mg/kg. Even in IgG4 format, HFB6-4hz1-hG4P is superior to 22B3-hG4P at 10 mg/kg. Furthermore, HFB6-4hz1-hG1 (10 mg/kg) treatment demonstrated long term survival benefit than Abatacept (6.13 mg/kg, molar equivalent to 10 mg/kg antibody), an approved prophylaxis to acute GvHD in human.

These data showed that the subject BTLA agonist antibody HFB6-4-hz1-hG1 is superior than the benchmark 22B3 agonist antibody in this mouse model of acute GvHD under the experimental condition, at least with respect to the better therapeutic efficacy throughout the course of the treatment, the overall prolonger and better survival rate, as well as reduced toxicity.

Overall, the data above demonstrated that the subject BTLA agonist antibodies, such as HFB6-4 and HFB6-5 and their derived humanized variants, have single-digit binding affinity to human and cyno BTLA; that multiple humanized variants of these antibodies (such as HFB6-4hz1-hG1) retained parental antibody function in B cell proliferation suppression and T cell reporter assay; that the antibodies exhibited favorable PK profiles in wile-type mice, supporting q3d or less frequent administration for efficacy evaluation. Further, in an in vivo hPBMC-NSG mouse model of acute GvHD, a subject humanized variant HFB6-4hz1-hG1 proved to be more efficacious and less toxic than a benchmark agonist antibody 22B3, even at doses one tenth ( 1/10) or one third (⅓) of that of the control antibody.

1. An isolated monoclonal antibody, or an antigen-binding fragment thereof, wherein said monoclonal antibody or antigen-binding fragment thereof is specific for and activates human BTLA (B- and T-Lymphocyte Attenuator) and optionally cross-reactive with cynomolgus BTLA, and wherein said monoclonal antibody comprises:
(1a) a heavy chain variable region (HCVR), comprising a HCVR CDR1 sequence of SEQ ID NO: 1, a HCVR CDR2 sequence of SEQ ID NO: 2, and a HCVR CDR3 sequence of SEQ ID NO: 3; and,
(1b) a light chain variable region (LCVR), comprising a LCVR CDR1 sequence of SEQ ID NO: 4, a LCVR CDR2 sequence of SEQ ID NO: 5, and a LCVR CDR3 sequence of SEQ ID NO: 6;
or,
(2a) a heavy chain variable region (HCVR), comprising a HCVR CDR1 sequence of SEQ ID NO: 61, a HCVR CDR2 sequence of SEQ ID NO: 62, and a HCVR CDR3 sequence of SEQ ID NO: 63; and,
(2b) a light chain variable region (LCVR), comprising a LCVR CDR1 sequence of SEQ ID NO: 64, a LCVR CDR2 sequence of SEQ ID NO: 65, and a LCVR CDR3 sequence of SEQ ID NO: 66;
optionally, said monoclonal antibody is not naturally occurring.

2. The isolated monoclonal antibody or antigen-binding fragment thereof of claim 1, wherein:
(1A) the HCVR sequence is SEQ ID NO: (7+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and,
(1B) the LCVR sequence is SEQ ID NO: (8+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region,
wherein n is any one of 1, 0, and 2-24; or,
(2A) the HCVR sequence is SEQ ID NO: (67+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in heavy chain framework region; and,
(2B) the LCVR sequence is SEQ ID NO: (68+2n), or a variant having at least 90%, 91%, 92%, 93%, 94%, 95%, 96%, 97%, 98%, 99% sequence identity thereto and comprising/consisting essentially of/consisting of at least one amino acid change (e.g., insertion, deletion, and/or substitution) in light chain framework region,
wherein n is any one of 0-24.

3. The isolated monoclonal antibody or antigen-binding fragment thereof according to claim 1, wherein said monoclonal antibody is an IgG1 antibody, an IgG2 antibody, an IgG3 antibody, or an IgG4 antibody.

4. (canceled)

5. The isolated monoclonal antibody or antigen-binding fragment thereof according to claim 3, wherein said IgG1 antibody comprises a heavy chain constant region sequence of SEQ ID NO: 57 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein, and a light chain constant region sequence of SEQ ID NO: 58 or a variant thereof having at least 90%, 91%, 92%, 93%, 94%, 95%, 96%, 97%, 98%, 99% sequence identity thereto and comprising an amino acid change (e.g., insertion, deletion, and/or substitution) therein.

6. The isolated monoclonal antibody or antigen-binding fragment thereof according to claim 1, wherein said monoclonal antibody is an IgG1 antibody comprising a mutation in the heavy chain constant region that:
(1) modulates antibody dependent cell mediated cytotoxicity (ADCC) and/or antibody dependent cell mediated phagocytosis (ADCP) (such as F243L, G236A, S239D/1332E, S239D/A330L/1332E, S298A/E333A/K334A, F243L/R292P/Y300L/V305I/P396L, or afucosylated (non-fucosylated) antibody (such as afucosylated N297 at Fc region) with enhanced ADCC through increased binding to FcγRIIIa); and/or,
(2) enhances serum half-life (such as T250Q/M428L, M252Y/S254T/T256E).

7. The isolated monoclonal antibody or antigen-binding fragment thereof according to claim 1, which is a mouse antibody, a human-mouse chimeric antibody, a humanized antibody, a human antibody, a CDR-grafted antibody, or a resurfaced antibody.

8. (canceled)

9. The isolated monoclonal antibody or antigen-binding fragment thereof according to claim 1, wherein said antigen-binding fragment thereof is an Fab, Fab′, F(ab′)₂, F_{d}, single chain Fv or scFv, disulfide linked Fv, V-NAR domain, IgNar, intrabody, IgGΔCH₂, minibody, F(ab′)₃, tetrabody, triabody, diabody, single-domain antibody, DVD-Ig, Fcab, mAb₂, (scFv)₂, or scFv-Fc.

10. The isolated monoclonal antibody or antigen-binding fragment thereof of claim 1, wherein said monoclonal antibody or antigen-binding fragment thereof cross-reacts with cynomolgus/rhesus monkey BTLA, but does not substantially cross-react with mouse BTLA;
wherein said monoclonal antibody or antigen-binding fragment thereof binds human BTLA with a K_{D} of less than about 25 nM, 20 nM, 15 nM, 10 nM, 5 nM, 2 nM, 1 nM, 0.5 nM or less;
wherein said monoclonal antibody or antigen binding fragment thereof is an agonist of human BTLA and activates downstream signaling from BTLA upon binding to BTLA, optionally said downstream signaling from BTLA inhibits B cell proliferation, and/or inhibits T cell (e.g., CD4, CD8, Th1, TFH, αβ, or γδ T-cell) and/or plasma cell activation;
wherein said isolated monoclonal antibody or antigen-binding fragment thereof does not block/interfere/abolish BTLA binding to HVEM;
wherein the said isolated monoclonal antibody or antigen-binding fragment thereof does not lead to significant weight loss when administered (e.g., i.p. to mouse) at a dose of 10 mg/kg BIW*6 (e.g., for 21 days);
wherein the said isolated monoclonal antibody or antigen-binding fragment thereof significantly reduces CD45⁺ T cells when administered in vivo (e.g., i.p. to mouse at a dose of 10 mg/kg BIW*6 for 14 days); and/or
wherein the said isolated monoclonal antibody or antigen-binding fragment thereof significantly reduces disease severity (e.g., mean total disease score of less than 3 at 3 weeks) and/or overall survival rate (e.g., overall survival rate of at least 80% at 4 weeks) in GvHD or an animal model thereof, compared to isotype matched control Ab.

11.-17. (canceled)

18. An isolated monoclonal antibody or an antigen-binding fragment thereof, which competes with the isolated monoclonal antibody or antigen-binding fragment thereof of claim 1 for binding to human BTLA.

19. The isolated monoclonal antibody or antigen-binding fragment thereof of claim 18, which inhibits B cell proliferation, and/or inhibits T cell (e.g., CD4, CD8, Th1, TFH, αβ, or γδ T-cell) activation, upon binding to BTLA.

20. The isolated monoclonal antibody or antigen-binding fragment thereof according to claim 1, comprising:
(1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or,
(2) the heavy chain amino acid sequence of SEQ ID NO: 59, and the light chain amino acid sequence of SEQ ID NO: 60.

21. An isolated monoclonal antibody or antigen-binding fragment thereof, comprising/consisting essentially of/consisting of:
(1) the HCVR sequence of SEQ ID NO: 9, the heavy chain constant region sequence of SEQ ID NO: 57, the LCVR sequence of SEQ ID NO: 10, and the light chain constant region sequence of SEQ ID NO: 58; or,
(2) the heavy chain amino acid sequence of SEQ ID NO: 59, and the light chain amino acid sequence of SEQ ID NO: 60.

22. A polynucleotide encoding the heavy chain or the light chain or the antigen-binding portion thereof of claim 1.

23. (canceled)

24. A vector comprising the polynucleotide of claim 22.

25. (canceled)

26. A pharmaceutical composition comprising the isolated monoclonal antibody or antigen-binding fragment thereof of claim 1.

27. The pharmaceutical composition of claim 26, which is formulated for intravenous (i.v.) infusion or administration, or for subcutaneous (s.c.) administration.

28. A method of down-regulating B-cell-mediated or T-cell-mediated immune response, or treating an autoimmune disorder, in a patient in need thereof, the method comprising administering to the patient an effective amount of the isolated monoclonal antibody or antigen-binding fragment thereof of claim 1.

29. The method of claim 28, which is for treating an autoimmune disorder.

30. The method of claim 29, wherein the autoimmune disorder is Systemic Lupus Erythematosus (SLE); ulcerative colitis (UC) including Pediatric Ulcerative Colitis; rheumatoid arthritis (RA); psoriasis (Ps) including Chronic Plaque Psoriasis; psoriac arthritis (PsA); Crohn's Disease (CD) including Pediatric Crohn's Disease; Inflammatory Bowel Disease (IBD); ankylosing spondylitis; Juvenile Idiopathic Arthritis (JIA) including Polyarticular Juvenile Idiopathic Arthritis; Hidradenitis Suppurativa; Non-Infectious Intermediate, Posterior, and Panuveitis; autoimmune hepatitis-like diseases; experimental autoimmune encephalomyelitis (EAE); MHC-mismatched cardiac allograft; inflammation of the lung in acute airway allergy; graft versus host disease (GvHD); or allogeneic hematopoietic stem cell transplantation (aHSCT).

31. The method of claim 28, further comprising administering to the patient a further agent effective to treat the autoimmune disorder.

32.-34. (canceled)
